(19) United States
(12) Patent Application Publication
(10) Pub. No.: US 2024/0300959 A1
(43) Pub. Date: Sep. 12, 2024

(54) BALASUBRAMIDE DERIVATIVE AND USE THEREOF IN PREPARATION OF MEDICINE FOR TREATING ACUTE LUNG INJURY

(71) Applicant: Guangdong Pharmaceutical University, Guangdong (CN)

(72) Inventors: Hansen LIN, Guangdong (CN); Tao PANG, Jiangsu (CN); Jinqian SONG, Jiangsu (CN)

(73) Assignee: Guangdong Pharmaceutical University, Guangdong (CN)

(21) Appl. No.: 18/603,126

(22) Filed: Mar. 12, 2024

Related U.S. Application Data
(63) Continuation of application No. PCT/CN2023/086315, filed on Apr. 4, 2023.

(30) Foreign Application Priority Data
Mar. 9, 2023 (CN) 202310227402.4

(51) Int. Cl. C07D 487/04; A61K 31/55; A61P 11/00

(52) U.S. Cl. CPC C07D 487/04; A61K 31/55; A61P 11/00

(57) ABSTRACT

Embodiments of the present invention belong to the technical field of biomedicine, and specifically relate to a balasubramide derivative and use thereof in preparation of a medicine for treating acute lung injury. According to the embodiments of the present invention, it is proven through experiments that the balasubramide derivative can significantly reduce the expression of TNF-α in a macrophage cell line inflammation model, has no obvious toxicity at an effective dose, and has high safety. Moreover, the balasubramide derivative (+) 3C-20 can significantly relieve sepsis or acute lung injury induced by lung exposure to bacterial endotoxin, achieves the effect of relieving the acute lung injury of mice by inhibiting the expression of inflammatory factors in macrophages, and significantly improves the survival rate of model mice with acute lung injury.

### CROSS-REFERENCE TO RELATED APPLICATION

This application is a continuation of international application of PCT application serial no. PCT/CN2023/086315, filed on Apr. 4, 2023, which claims the priority benefit of China application no. 202310227402.4, filed on Mar. 9, 2023. The entirety of each of the above mentioned patent applications is hereby incorporated by reference herein and made a part of this specification.

### TECHNICAL FIELD

The present invention belongs to the technical field of biomedicine. More specifically, the present invention relates to a balasubramide derivative and use thereof in preparation of a medicine for treating acute lung injury.

### RELATED ART

Sepsis is a severe syndrome caused by invasion of bacteria and other pathogenic microorganisms into the body, which has the clinical manifestation of systemic inflammatory response syndrome, accompanied by septicopyemia, insufficient blood perfusion of organs and other complications. In addition, the sepsis will also lead to septic shock, acute lung injury (ALI)/acute respiratory distress syndrome (ARDS), multiple organ failure and even death. At present, it is believed that uncontrolled inflammatory response and immune disorder caused by infection of the pathogenic microorganisms in the body are main causes of the sepsis. During infection with the sepsis, endotoxin and the like stimulate immune cells to produce a large number of inflammatory mediators which prompt the inflammatory response cells, especially macrophages, to be recruited and activated in tissues to further produce cytokines, chemokines, oxygen free radicals and the like, thereby forming a waterfall-like cascade reaction, so that damage and even failure of organs, such as the lung, are caused, and finally, death of patients is caused.

The acute lung injury (ALI) is a common serious disease in clinical practice. The ALI is usually developed into the acute respiratory distress syndrome (ARDS), where the latter one has the obvious characteristics of pulmonary edema, reduced lung compliance and acute hypoxic respiratory failure, eventually leading to death. According to statistics, North America has about 150 thousand ARDS patients every year. Although the mortality rate has been significantly reduced due to mechanical ventilation and other auxiliary medical equipment, 40%-70% of patients still die from the ARDS, and the mortality rate is as high as 90% when the disease is accompanied by the sepsis and other complications, wherein the sepsis is the most common cause of the ALI/ARDS. The ALI is usually believed to have a pathophysiological mechanism related to lung inflammation disorder, and has the pathological characteristics of increased production of preinflammatory factors, increased infiltration of inflammatory cells and apoptosis of alveolar epithelial cells, leading to lung injury.

As an eight-membered lactam compound extracted from leaves of Clausena indica (Dalz.) Oliv of rutaceae in Sri Lanka, balasubramide is subjected to structural modification to obtain (+)3C-20 (CN110684027A) having a significant therapeutic effect on neuroinflammation caused by brain injury.

### SUMMARY OF INVENTION

The technical problems to be solved by the present invention are to overcome the defects and disadvantages that current diseases, such as acute lung injury, have a high mortality rate and better treatment means are in shortage, and to provide a balasubramide derivative.

Another purpose of the present invention is to provide use of a balasubramide derivative in preparation of an anti-inflammatory drug.

Another purpose of the present invention is to provide use of a balasubramide derivative in preparation of a medicine for treating acute lung injury.

The above purposes of the present invention are realized by the following technical schemes:

A balasubramide derivative is provided. The balasubramide derivative has a structure shown in Formula (I):

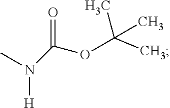

where R is hydrogen, halogen, C₁₋₄ alkyl, C₁₋₇ alkoxyl, benzyloxy or nitro-substituted benzoyloxy; R₁ is hydrogen or

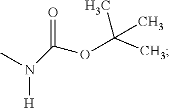

R₂ is hydrogen, C₁₋₄ alkyl or C₂₋₅ alkenyl; and the R, the R₁ and the R₂ are not hydrogen simultaneously.

Preferably, the R is hydrogen, halogen, C₁₋₃ alkyl, C₁₋₅ alkoxyl, benzyloxy or nitro-substituted benzoyloxy; the R₁ is hydrogen or

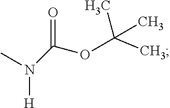

the R₂ is hydrogen, C₁₋₃ alkyl or C₂₋₃ alkenyl; and the R, the R₁ and the R₂ are not hydrogen simultaneously.

More preferably, the R is hydrogen, halogen, methyl, ethoxyl, benzyloxyl or nitro-substituted benzoyloxy; the R₁ is hydrogen or

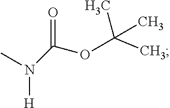

the R₂ is hydrogen, methyl, ethyl, propyl or allyl; and the R, the R₁ and the R₂ are not hydrogen simultaneously.

Additionally, the present invention further provides use of a balasubramide derivative in preparation of an anti-inflammatory drug. The balasubramide derivative has a structure shown in Formula (I):

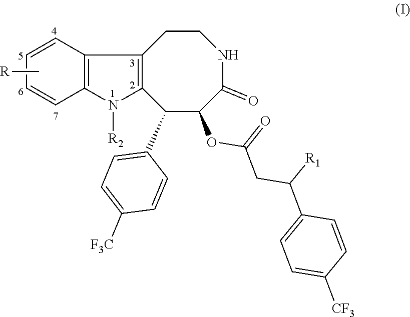

where R is hydrogen, halogen, C₁₋₄ alkyl, C₁₋₇ alkoxyl, benzyloxy or nitro-substituted benzoyloxy; R₁ is hydrogen or

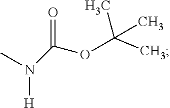

and R₂ is hydrogen, C₁₋₄ alkyl or C₂₋₅ alkenyl.

Additionally, the present invention further provides use of a balasubramide derivative in preparation of a medicine for treating acute lung injury. The balasubramide derivative has a structure shown in Formula (I):

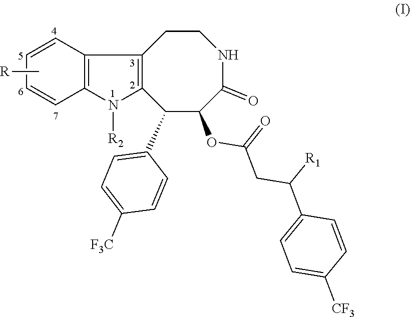

where R is hydrogen, halogen, C₁₋₄ alkyl, C₁₋₇ alkoxyl, benzyloxy or nitro-substituted benzoyloxy; R₁ is hydrogen or

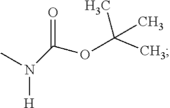

and R₂ is hydrogen, C₁₋₄ alkyl or C₂₋₅ alkenyl.

Further, the acute lung injury is caused by sepsis.

Further, the acute lung injury is induced by bacterial endotoxin. Preferably, the bacterial endotoxin is lipopolysaccharide.

Further, the acute lung injury includes septicopyemia caused by peripheral inflammation, inflammatory lung injury, bronchial asthma, tracheitis, bronchitis, chronic obstructive pulmonary disease, pulmonary heart disease and pulmonary fibrosis.

Further, the balasubramide derivative treats the acute lung injury by inhibiting the expression of inflammatory factors. Further, the inflammatory factors include TNF-α, IL-1β, IL-6 and COX-2.

Further, the medicine includes an effective amount of a balasubramide derivative or a pharmaceutically acceptable salt, solvate, enantiomer, diastereoisomer or tautomer thereof.

Furthermore, the medicine includes an effective amount of a balasubramide derivative and a pharmaceutically acceptable excipient. Further, the medicine has a dosage form of a pulmonary or nasal inhalation nebulizer, a quantitative aerosol or a dry powder inhaler.

The present invention has the following beneficial effects:

According to the present invention, it is proven through experiments that the balasubramide derivative can significantly reduce the expression of TNF-α in a macrophage cell line inflammation model, has no obvious toxicity at an effective dose, and has high safety. Moreover, the balasubramide derivative (+) 3C-20 can significantly relieve sepsis or acute lung injury induced by lung exposure to bacterial endotoxin, achieves the effect of relieving the acute lung injury of mice by inhibiting the expression of inflammatory factors in macrophages, and significantly improves the survival rate of model mice with acute lung injury.

### BRIEF DESCRIPTION OF DRAWINGS

FIG. 1 shows data statistics of the influence of balasubramide derivatives with different structures at a concentration of 10 μM on the expression and release of an inflammatory factor TNF-α in RAW264.7 macrophages stimulated by lipopolysaccharide.

FIG. 2 shows data statistics of the influence of balasubramide derivatives with different structures at a concentration of 10 μM on the viability of RAW264.7 cells.

FIG. 3 shows data statistics of the influence of (+)3C-20 with different concentrations on the expression and release of an inflammatory factor TNF-α in RAW264.7 macrophages stimulated by lipopolysaccharide.

FIG. 4 shows data statistics of the influence of (+)3C-20 with different concentrations on the expression level of inflammation genes in RAW264.7 macrophages stimulated by lipopolysaccharide.

FIG. 5 shows data statistics of the influence of (+)3C-20 with different concentrations on the expression and release of an inflammatory factor TNF-α in bone marrow derived macrophages (BMDMs) stimulated by lipopolysaccharide.

FIG. 6 shows data statistics of the influence of (+)3C-20 on the body weight and lung index of mice with sepsis and lung injury caused by intraperitoneal injection of lipopolysaccharide.

FIG. 7-1 and FIG. 7-2 show data statistics of the influence of (+)3C-20 on the expression of an inflammatory factor TNF-α in the serum of mice with sepsis and lung injury caused by intraperitoneal injection of lipopolysaccharide and pathological sections of lung tissues.

FIG. 8 shows data statistics of the influence of (+)3C-20 on the mRNA expression level of inflammation genes in lung tissues of mice with sepsis and lung injury caused by intraperitoneal injection of lipopolysaccharide.

FIG. 9 shows data statistics of the influence of (+)3C-20 on the body weight and lung index of mice with acute lung injury caused by intratracheal instillation of lipopolysaccharide.

FIG. 10-1 and FIG. 10-2 show data statistics of the influence of (+)3C-20 on the expression of an inflammatory factor TNF-α in lung tissues of mice with acute lung injury caused by intratracheal instillation of lipopolysaccharide and pathological sections of lung tissues.

FIG. 11-1 and FIG. 11-2 show data statistics of the influence of (+)3C-20 on the mRNA expression level of inflammation genes in lung tissues of mice with acute lung injury caused by intratracheal instillation of lipopolysaccharide.

FIG. 12 shows data statistics of the influence of (+)3C-20 on the lung wet-dry weight ratio, the level of TNF-α in bronchoalveolar lavage fluid, the total protein and the ratio of immune myeloid cells of mice with acute lung injury caused by intratracheal instillation of lipopolysaccharide.

FIG. 13 shows data statistics of the influence of (+)3C-20 on the survival rate of mice with acute lung injury caused by intratracheal instillation of lipopolysaccharide.

### DESCRIPTION OF EMBODIMENTS

The present invention is further explained below in combination with drawings attached to the specification and specific examples, but the examples are not intended to limit the present invention in any manner. Unless otherwise specified, reagents, methods and equipment used in the present invention are conventional reagents, methods and equipment in the technical field.

Lipopolysaccharide (LPS) was purchased from Beyotime Biotechnology Co., Ltd., and other reagents were commercially available analytical reagents. Cells: An RAW264.7 macrophage cell line was purchased from China Center for Type Culture Collection. Animals: ICR mice and C57BL/6J mice were purchased from Nanjing Huachuang Biotechnology Co., Ltd. Kits: An ELISA kit of TNF-α was purchased from Shenzhen Dakewe Biotech Co., Ltd., an RNA extraction kit and an SYBR reagent were purchased from Vazyme Biotech Co., Ltd., a primer was purchased from Genewiz Biotechnology Co., Ltd., and a flow antibody was purchased from Nanjing Fcmacs Biotech Co., Ltd.

Balasubramide derivatives (+)-3C, (+)-3C-20, (±)-3C-20 and (+)-B001 were synthesized and prepared with reference to a Chinese patent application No. CN110684027A.

A method for preparing other balasubramide derivatives is as follows:

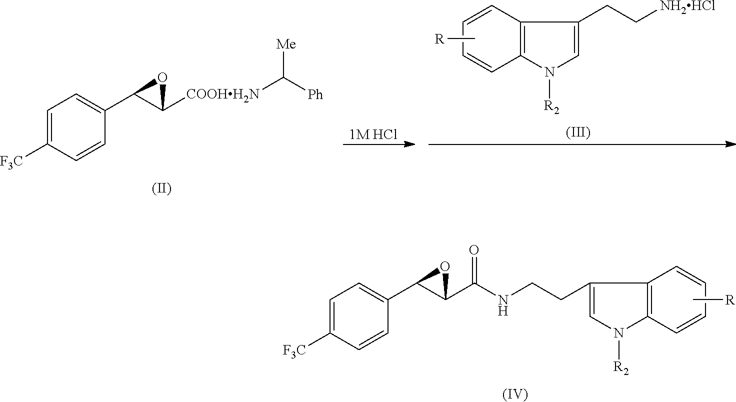

Step S1, 3.53 g (10 mmol) of a compound shown in Formula(+)-(II) was weighed and dissolved in 30 mL of distilled water, 30 mL of ethyl acetate was added, the pH value was adjusted to 3-4 with 1 M HCl under an ice bath, and standing was performed for layering. Aqueous layer was extracted with 10 mL of ethyl acetate for 3 times, and organic layers were combined, washed with 15 mL of a saturated sodium chloride aqueous solution, dried and concentrated. 30 mL of DMF was added, 1.62 g (10 mmol) of CDI was added under an ice bath, and a resulting mixture was heated to room temperature and stirred for 1 h. 3.03 g (30 mmol) of triethylamine and 10 mmol of a compound shown in Formula (III) were added under stirring to carry out a reaction at room temperature for 8 h. After the reaction was completed, 50 mL of distilled water was added, extraction was performed with 30 mL of ethyl acetate for 3 times, and organic layers were combined, washed with 30 mL of a saturated sodium chloride aqueous solution for 3 times, dried and concentrated. 10 mL of n-hexane was added for beating, evaporation was performed under reduced pressure to remove a solvent, and a resulting solid was recrystallized with ethyl acetate and n-hexane to obtain a compound shown in Formula (IV).

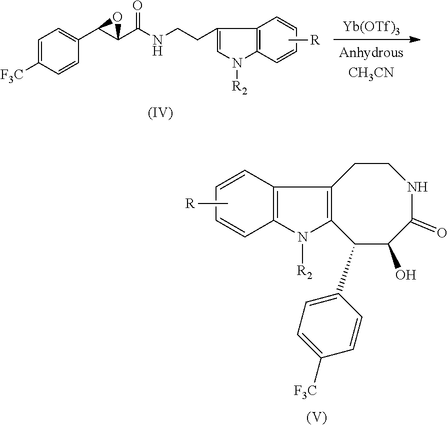

Step S2, at room temperature, 6.0 mmol of the compound shown in Formula (IV), 0.744 g (1.2 mmol) of ytterbium trifluoromethanesulfonate and 20 mL of anhydrous acetonitrile were added into a 50 mL eggplant-shaped flask under stirring to carry out a reaction at room temperature for 8 h. Concentration was performed, a residue was dissolved in 30 mL of dichloromethane, washed with 20 mL of a saturated sodium chloride aqueous solution for 2 times, dried and concentrated, and then the residue was purified by column chromatography to obtain a compound shown in Formula (V).

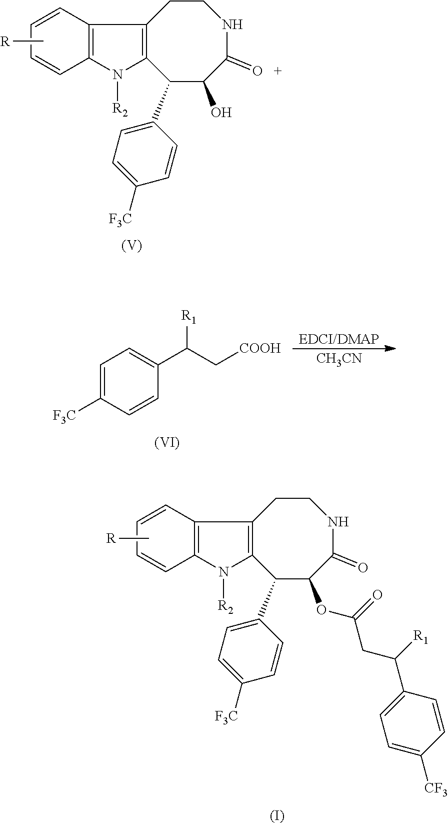

Step S3, at room temperature, 3 mmol of the compound shown in Formula (V) was dissolved in 10 mL of acetonitrile in a 50 mL eggplant-shaped flask, and 0.690 g (3.6 mmol) of EDCI, 81 mg (0.6 mmol) of DMAP and 0.655 g (3 mmol) of a compound shown in Formula (VI) were sequentially added under stirring and then stirred to carry out a reaction for 3 h. After the reaction was completed, concentration was performed, a residue was dissolved in 30 mL of dichloromethane, sequentially washed with 10 mL of a saturated sodium bicarbonate aqueous solution and 10 mL of a saturated sodium chloride aqueous solution, dried and concentrated, and then the residue was purified by column chromatography. A resulting solid was recrystallized with a mixture of n-hexane and ethyl acetate at a volume ratio of 1:1 to obtain a compound shown in Formula (I).

Structures and related data of the obtained balasubramide derivatives are as follows:

1. (+)-B3a: C₃₀H₂₃F₇N₂O₃, 592.1597, White powder 1.39 g, yield 78.1%, m.p.222.9-223.4° C., [α]_{D}²⁰=+14.6 (c 0.5, CH₃OH); ¹H NMR (600 Hz, Chloroform-d) δ 8.17 (s, 1H), 7.56 (d, J=8.20 Hz, 2H), 7.50 (d, J=8.13 Hz, 2H), 7.42 (d, J=8.23 Hz, 2H), 7.19 (d, J=7.99 Hz, 2H), 7.06 (td, J=7.98, 4.96 Hz, 1H), 7.00 (d, J=8.01 Hz, 1H), 6.76 (dd, J=11.41, 7.76 Hz, 1H), 5.67 (d, J=11.14 Hz, 1H), 4.68 (d, J=11.18 Hz, 1H), 3.96-3.85 (m, 2H), 3.51-3.37 (m, 2H), 2.93 (t, J=7.72 Hz, 2H), 2.78-2.61 (m, 2H).

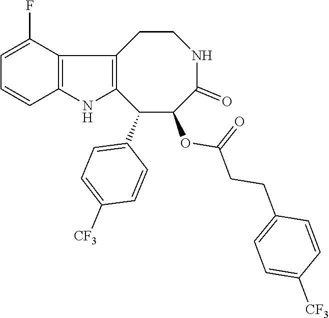 [SMILES: O=C(CCc1ccc(C(F)(F)F)cc1)O[C@@H]1C(=O)NCCc2c([nH]c3cccc(F)c23)[C@H]1c1ccc(C(F)(F)F)cc1]

2. (+)-B3c: C₃₁H₂₆F₆N₂O₄, 604.1797, White powder 1.44 g, yield 79.5%, m.p.272.6-274.7° C., [α]_{D}²⁰=+13.1 (c 0.5, CH₃OH); ¹H NMR (600 Hz, Chloroform-d) δ 7.96 (s, 1H), 7.53 (d, J=8.15 Hz, 2H), 7.49 (d, J=7.91 Hz, 2H), 7.38 (d, J=8.15 Hz, 2H), 7.17 (d, J=7.96 Hz, 1H), 7.12 (d, J=8.73 Hz, 1H), 6.89 (d, J=2.25 Hz, 1H), 6.83 (dd, J=8.74, 2.37 Hz, 1H), 5.66 (d, J=11.16 Hz, 1H), 4.68 (d, J=11.07 Hz, 1H), 3.98-3.94 (m, 1H), 3.86 (s, 3H), 3.52-3.42 (m, 2H), 3.40-3.35 (m, 1H), 2.91 (t, J=7.63 Hz, 2H), 2.77-2.60 (m, 2H).

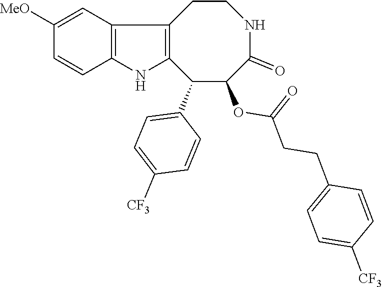 [SMILES: COc1ccc2[nH]c3c(c2c1)CCNC(=O)[C@@H](OC(=O)CCc1ccc(C(F)(F)F)cc1)[C@@H]3c1ccc(C(F)(F)F)cc1]

3. (+)-B3d: C₃₀H₂₃F₇N₂O₃, 592.1597, White powder 1.37 g, yield 76.9%, m.p.147.3-149.2° C., [α]_{D}²°=+13.0 (c 0.5, CH₃OH); ¹H NMR (600 Hz, Chloroform-d) δ 7.98 (s, 1H), 7.59 (d, J=8.21 Hz, 2H), 7.52-7.47 (m, 3H), 7.39 (d, J=8.11 Hz, 1H), 7.30 (d, J=8.10 Hz, 1H), 7.25-7.22 (m, 1H), 7.16 (d, J=7.99 Hz, 1H), 6.96-6.85 (m, 2H), 6.86-6.77 (m, 1H), 5.70 (d, J=3.95 Hz, 1H), 4.47-4.46 (m, 1H), 3.54-3.28 (m, 3H), 2.99 (t, J=7.46 Hz, 1H), 2.73-2.69 (m, 2H), 2.62-2.43 (m, 2H).

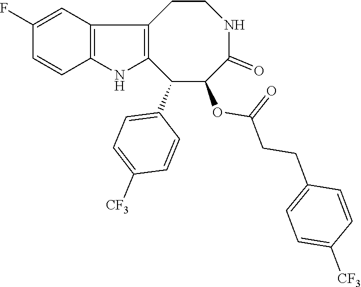 [SMILES: O=C(CCc1ccc(C(F)(F)F)cc1)O[C@@H]1C(=O)NCCc2c([nH]c3ccc(F)cc23)[C@H]1c1ccc(C(F)(F)F)cc1]

4. (+)-B3e: C₃₀H₂₃BrF₆N₂O₃, 652.0796, White powder 1.58 g, yield 80.8%, m.p.226.1-227.3° C., [α]_{D}²⁰=+14.2 (c 0.5, CH₃OH); ¹H NMR (600 Hz, Chloroform-d) δ 8.07 (s, 1H), 7.67 (d, J=1.51 Hz, 1H), 7.55 (d, J=7.90 Hz, 2H), 7.37 (d, J=8.20 Hz, 2H), 7.49 (d, J=8.08 Hz, 2H), 7.25 (d, J=1.80 Hz, 1H), 7.18 (d, J=8.01 Hz, 2H), 7.10 (d, J=8.52 Hz, 1H), 5.65 (d, J=11.13 Hz, 1H), 4.69 (d, J=11.15 Hz, 1H), 3.50-3.44 (m, 2H), 3.39-3.36 (m, 1H), 2.92 (t, J=7.63 Hz, 2H), 2.78-2.61 (m, 3H).

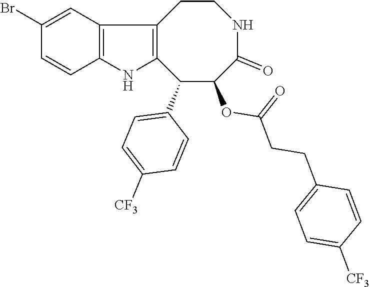 [SMILES: O=C(CCc1ccc(C(F)(F)F)cc1)O[C@@H]1C(=O)NCCc2c([nH]c3ccc(Br)cc23)[C@H]1c1ccc(C(F)(F)F)cc1]

5. (+)-B3f: C₃₀H₂₃ClF₆N₂O₃, 608.1301, White powder 1.46 g, yield 79.8%, m.p.301.9-302.7° C., [α]_{D}²⁰=+13.7 (c 0.5, CH₃OH); ¹H NMR (600 Hz, Chloroform-d) δ 8.02 (s, 1H), 7.55 (d, J=8.22 Hz, 2H), 7.49 (d, J=8.08 Hz, 2H), 7.44 (d, J=8.49 Hz, 1H), 7.38 (d, J=8.23 Hz, 2H), 7.22 (d, J=1.63 Hz, 1H), 7.18 (d, J=7.98 Hz, 2H), 7.11 (dd, J=8.52, 1.78 Hz, 1H), 5.66 (d, J=11.14 Hz, 1H), 4.6 8 (d, J=11.15 Hz, 1H), 4.01-3.97 (m, 1H), 3.53-3.36 (m, 3H), 2.92 (t, J=7.66 Hz, 2H), 2.78-2.61 (m, 2H).

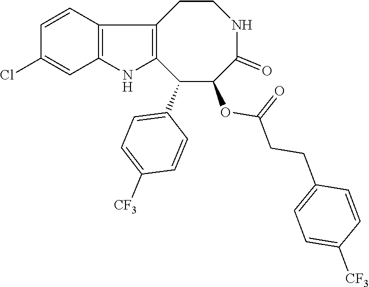 [SMILES: O=C(CCc1ccc(C(F)(F)F)cc1)O[C@@H]1C(=O)NCCc2c([nH]c3cc(Cl)ccc23)[C@H]1c1ccc(C(F)(F)F)cc1]

6. (+)-B3g: C₃₁H₂₆F₆N₂O₃, 588.1848, White powder 1.38 g, yield 78.2%, m.p.189.1-190.5° C., [α]_{D}²⁰=+13.7 (c 0.5, CH₃OH); ¹H NMR (600 Hz, Chloroform-d) δ 7.99 (s, 1H), 7.55 (d, J=8.18 Hz, 2H), 7.49 (d, J=8.09 Hz, 2H), 7.41 (dd, J=12.91, 8.20 Hz, 3H), 7.18 (d, J=8.01 Hz, 2H), 7.07 (t, J=7.28 Hz, 1H), 6.98 (d, J=7.10 Hz, 1H), 5.66 (d, J=11.15 Hz, 1H), 4.75 (d, J=11.12 Hz, 1H), 3.99-3.95 (m, 1H), 3.58-3.53 (m, 1H), 3.49-3.36 (m, 2H), 2.93 (t, J=7.69 Hz, 2H), 2.79-2.62 (m, 2H), 2.36 (s, 3H).

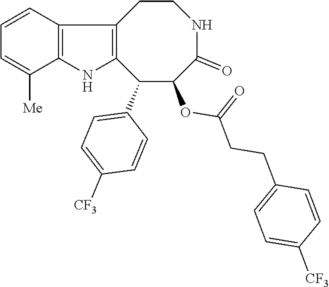 [SMILES: Cc1cccc2c3c([nH]c12)[C@@H](c1ccc(C(F)(F)F)cc1)[C@H](OC(=O)CCc1ccc(C(F)(F)F)cc1)C(=O)NCC3]

7. (+)-B3h: C₃₇H₃₀F₆N₂O₄, 680.2110, White powder 1.63 g, yield 79.7%, m.p.118.9-120.1° C., [α]_{D}²⁰=+14.5 (c 0.5, CH₃OH); ¹H NMR (600 Hz, Chloroform-d) δ 8.02 (s, 1H), 7.53 (d, J=8.2 Hz, 2H), 7.48 (t, J=7.7 Hz, 4H), 7.41-7.36 (m, 4H), 7.33 (t, J=7.4 Hz, 1H), 7.16 (d, J=8.0 Hz, 2H), 7.12 (d, J=8.7 Hz, 1H), 7.07 (d, J=2.3 Hz, 1H), 6.91 (dd, J=8.7, 2.4 Hz, 1H), 5.66 (s, 1H), 5.11 (s, 2H), 4.68 (d, J=11.1 Hz, 1H), 3.97-3.90 (m, 1H), 3.49-3.33 (m, 3H), 2.91 (t, J=7.7 Hz, 2H), 2.76-2.60 (m, 2H).

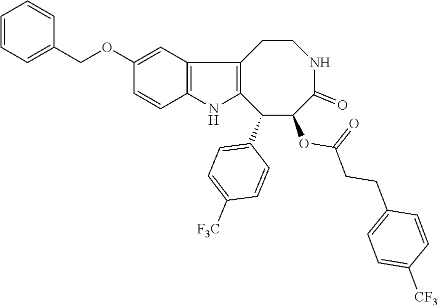 [SMILES: O=C(CCc1ccc(C(F)(F)F)cc1)O[C@@H]1C(=O)NCCc2c([nH]c3ccc(OCc4ccccc4)cc23)[C@H]1c1ccc(C(F)(F)F)cc1]

8. (+)-c1: C₃₁H₂₆F₆N₂O₃, 588.18, White solid, 78.3% yield, mp.234-236° C., [α]20 D[α]_{D}²⁰=+13.6 (c 0.5, CH₃OH), ¹H NMR (600 MHz, CDCl₃) δ 7.57 (dd, J=12.1, 8.1 Hz, 3H), 7.51 (d, J=8.1 Hz, 2H), 7.41 (d, J=8.1 Hz, 2H), 7.24 (d, J=3.8 Hz, 2H), 7.20 (d, J=8.0 Hz, 2H), 7.16 (dd, J=8.0, 4.0 Hz, 1H), 5.87 (d, J=5.1 Hz, 1H), 5.70 (d, J=11.2 Hz, 1H), 4.80 (d, J=11.2 Hz, 1H), 4.00-3.92 (m, 1H), 3.66-3.57 (m, 1H), 3.51-3.39 (m, 5H), 2.95 (t, J=7.7 Hz, 2H), 2.78 (dt, J=15.6, 7.7 Hz, 1H), 2.69-2.63 (m, 1H).

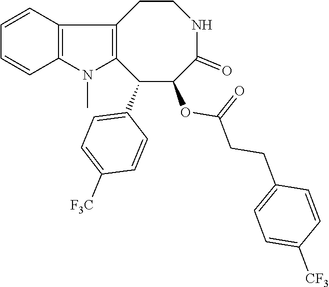 [SMILES: Cn1c2c(c3ccccc31)CCNC(=O)[C@@H](OC(=O)CCc1ccc(C(F)(F)F)cc1)[C@@H]2c1ccc(C(F)(F)F)cc1]

9. (+)-c2: C₃₂H₂₈F₆N₂O₃, 602.20, White solid, 70.6% yield, mp.245-247° C., [α]20 D[α]_{D}²⁰=+14.2 (c 0.5, CH₃OH), ¹H NMR (600 MHz, CDCl₃) δ 7.59 (d, J=7.9 Hz, 1H), 7.55 (d, J=8.2 Hz, 2H), 7.51 (d, J=8.1 Hz, 2H), 7.41 (d, J=8.2 Hz, 2H), 7.25 (d, J=5.7 Hz, 1H), 7.22 (dd, J=9.8, 4.3 Hz, 3H), 7.18-7.14 (m, 1H), 5.83 (d, J=4.7 Hz, 1H), 5.68 (d, J=11.1 Hz, 1H), 4.77 (d, J=11.1 Hz, 1H), 4.02 (dq, J=14.6, 7.2 Hz, 1H), 3.95 (dd, J=12.8, 3.8 Hz, 1H), 3.90 (td, J=14.5, 7.2 Hz, 1H), 3.65-3.56 (m, 1H), 3.4-3.39 (m, 2H), 2.96 (t, J=7.7 Hz, 2H), 2.79 (dt, J=15.5, 7.6 Hz, 1H), 2.71-2.64 (m, 1H), 1.03 (t, J=7.2 Hz, 3H).

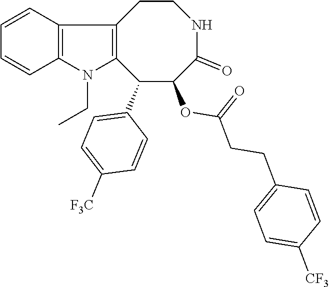 [SMILES: CCn1c2c(c3ccccc31)CCNC(=O)[C@@H](OC(=O)CCc1ccc(C(F)(F)F)cc1)[C@@H]2c1ccc(C(F)(F)F)cc1]

10. (+)-c3: C₃₃H₃₀F₆N₂O₃, 616.21, White solid, 68.5% yield, mp.242-245° C., [α]20 D[α]_{D}²⁰=+17.3 (c 0.5, CH₃OH), ¹H NMR (600 MHz, CDCl₃) δ 7.58 (d, J=7.9 Hz, 1H), 7.55 (d, J=7.9 Hz, 2H), 7.51 (d, J=7.7 Hz, 2H), 7.40 (d, J=7.9 Hz, 2H), 7.27-7.19 (m, 4H), 7.16 (d, J=7.4 Hz, 1H), 5.72 (d, J=5.0 Hz, 1H), 5.67 (d, J=11.0 Hz, 1H), 4.77 (d, J=11.0 Hz, 1H), 3.91 (ddd, J=15.7, 15.1, 7.5 Hz, 2H), 3.79-3.70 (m, 1H), 3.66-3.57 (m, 1H), 3.47-3.37 (m, 2H), 2.96 (t, J=7.6 Hz, 2H), 2.84-2.76 (m, 1H), 2.72-2.64 (m, 1H), 1.64-1.57 (m, 1H), 1.35 (dd, J=14.9, 7.8 Hz, 1H), 0.81 (t, J=7.3 Hz, 3H).

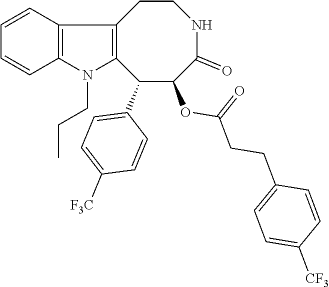 [SMILES: CCCn1c2c(c3ccccc31)CCNC(=O)[C@@H](OC(=O)CCc1ccc(C(F)(F)F)cc1)[C@@H]2c1ccc(C(F)(F)F)cc1]

11. (+)-c4: C₃₃H₂₈F₆N₂O₃, 614.20, White solid, 67.5% yield, mp.251-253° C., [α] 20 D=+16.2 (c 0.5, CH₃OH), ¹H NMR (600 MHz, CDCl₃) δ 7.58 (d, J=7.8 Hz, 1H), 7.52 (dd, J=22.0, 8.1 Hz, 4H), 7.38 (d, J=8.1 Hz, 2H), 7.22-7.14 (m, 5H), 6.16 (s, 1H), 5.66 (d, J=11.2 Hz, 1H), 5.6-5.55 (m, 1H), 4.75 (d, J=10.3 Hz, 1H), 4.69 (d, J=11.2 Hz, 1H), 4.55 (ddd, J=23.4, 12.8, 10.7 Hz, 1H), 4.48 (d, J=17.1 Hz, 1H), 4.36-4.26 (m, 1H), 3.98-3.87 (m, 1H), 3.68-3.55 (m, 1H), 3.50-3.38 (m, 2H), 2.93 (t, J=7.7 Hz, 2H), 2.81-2.59 (m, 2H).

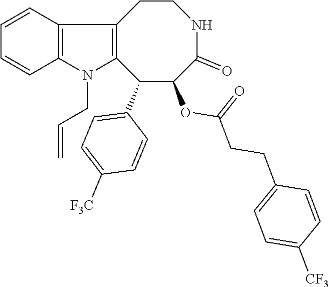 [SMILES: C=CCn1c2c(c3ccccc31)CCNC(=O)[C@@H](OC(=O)CCc1ccc(C(F)(F)F)cc1)[C@@H]2c1ccc(C(F)(F)F)cc1]

12. (+)-c5: C₃₅H₃₃F₆N₃O₅, 689.23, White solid 0.12 g, 32.6% yield, mp.189-192° C., [α]_{D}²⁰=+89.6 (c 1, CH3OH), ¹H NMR (600 MHz, CDCl₃) δ 8.49 (s, 1H), 7.51 (d, J=8.6 Hz, 3H), 7.44 (d, J=8.0 Hz, 2H), 7.32 (d, J=7.6 Hz, 2H), 7.17 (d, J=8.0 Hz, 3H), 7.14-7.11 (m, 2H), 5.87 (s, 1H), 5.76 (s, 1H), 5.55 (d, J=8.8 Hz, 1H), 4.73 (d, J=11.0 Hz, 1H), 3.82 (d, J=10.5 Hz, 1H), 3.48 (dd, J=13.7, 8.3 Hz, 1H), 3.31 (ddd, J=23.7, 15.0, 8.1 Hz, 2H), 2.84 (s, 2H), 1.42 (s, 9H).

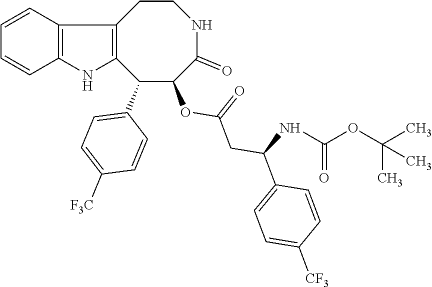 [SMILES: CC(C)(C)OC(=O)N[C@H](CC(=O)O[C@@H]1C(=O)NCCc2c([nH]c3ccccc23)[C@H]1c1ccc(C(F)(F)F)cc1)c1ccc(C(F)(F)F)cc1]

13. (+)-D7a: C₃₂H₂₈F₆N₂O₄, 618.19, White solid 30 mg, 43.5% yield. m.p.239.6-242.1° C., [α]_{D}²⁰=+21.8 (c 0.5, MeOH); ¹H NMR (600 MHz, Chloroform-d) δ 8.16 (s, 1H), 7.56 (dd, J=23.5, 8.9 Hz, 4H), 7.36 (d, J=7.6 Hz, 2H), 7.14 (d, J=7.7 Hz, 2H), 7.08 (d, J=9.5 Hz, 1H), 6.96 (d, J=3.5 Hz, 1H), 6.85 (dd, J=9.9, 1.9 Hz, 1H), 5.60 (d, J=10.9 Hz, 1H), 4.66 (d, J=10.3 Hz, 1H), 4.13 (q, J=8.2 Hz, 2H), 3.54-3.35 (m, 4H), 2.91 (t, J=6.9 Hz, 2H), 2.74-2.58 (m, 2H), 1.43 (t, J=6.8 Hz, 3H).

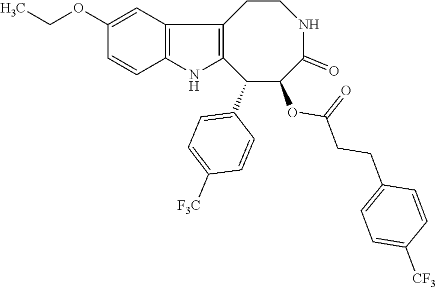 [SMILES: CCOc1ccc2[nH]c3c(c2c1)CCNC(=O)[C@@H](OC(=O)CCc1ccc(C(F)(F)F)cc1)[C@@H]3c1ccc(C(F)(F)F)cc1]

14. (+)-D7c: 739.62, Yellow solid 94.1 mg, 74.9% yield. m.p.225.2-227.9° C., [α]_{D}²⁰=+19.8 (c 0.5, CHCl₃); ¹H NMR (600 MHz, Chloroform-d) δ 8.54 (s, 1H), 8.42-8.33 (m, 4H), 7.53 (d, J=8.1 Hz, 2H), 7.47 (d, J=8.0 Hz, 2H), 7.38 (d, J=8.1 Hz, 2H), 7.30 (d, J=2.2 Hz, 1H), 7.11 (dd, J=13.9, 8.3 Hz, 3H), 6.92 (dd, J=8.6, 2.2 Hz, 1H), 5.67 (d, J=11.2 Hz, 1H), 4.71 (d, J=11.2 Hz, 1H), 3.90 (t, J=10.4 Hz, 1H), 3.39-3.28 (m, 3H), 2.87 (t, J=7.8 Hz, 2H), 2.72-2.57 (m, 2H).

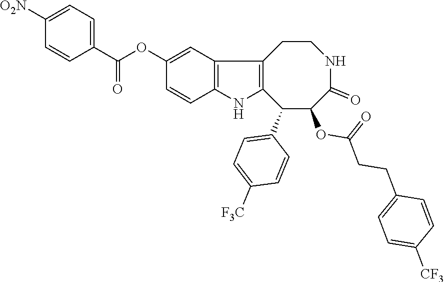 [SMILES: O=C(CCc1ccc(C(F)(F)F)cc1)O[C@@H]1C(=O)NCCc2c([nH]c3ccc(OC(=O)c4ccc([N+](=O)[O-])cc4)cc23)[C@H]1c1ccc(C(F)(F)F)cc1]

15. (+)-3C: C₂₀H₁₇F₃N₂O₂, 374.12, [α]_{D}²=+16.8 (c 0.5, CHCl₃)¹H NMR (600 Hz, DMSO-d₆) δ 10.91 (s, 1H), 7.65 (d, J=8.27 Hz, 2H), 7.54 (dd, J=14.17, 8.21 Hz, 3H), 7.34 (dd, J=6.92, 4.17 Hz, 1H), 7.18 (d, J=7.94 Hz, 1H), 7.03-7.00 (m, 1H), 6.98-6.95 (m, 1H), 5.21 (d, J=9.34 Hz, 1H), 4.89 (t, J=9.83 Hz, 1H), 4.30 (d, J=10.25 Hz, 1H), 3.7-3.65 (m, 1H), 3.28-3.18 (m, 2H).

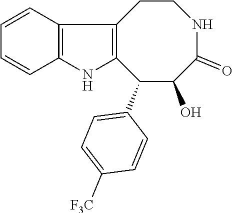 [SMILES: O=C1NCCc2c([nH]c3ccccc23)[C@@H](c2ccc(C(F)(F)F)cc2)[C@@H]1O]

16. (+)-B001: C₁₉H₁₇BrH₂O₂, 385.25, [α]_{D}²⁰=+20.8 (c 0.5, CHCl₃)¹H NMR (500 MHz, Chloroform-d) δ 9.47 (s, 1H), 7.66 (d, J=1.5 Hz, 1H), 7.50 (t, J=7.1 Hz, 1H), 7.36-7.23 (m, 4H), 7.19-7.12 (m, 3H), 5.82 (d, J=7.7 Hz, 1H), 4.97 (dd, J=7.7, 7.0 Hz, 1H), 4.54 (dd, J=7.0, 0.9 Hz, 1H), 3.61-3.47 (m, 2H), 2.93 (td, J=7.1, 2.2 Hz, 2H).

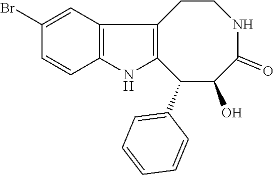 [SMILES: O=C1NCCc2c([nH]c3ccc(Br)cc23)[C@@H](c2ccccc2)[C@@H]1O]

17. (±)-3C-20: C₃₀H₂₄F₆N₂O₃, 574.16, ¹H NMR (600 Hz, Chloroform-d) δ 7.80 (s, 1H), 7.57 (d, J=7.75 Hz, 1H), 7.51 (dd, J=20.54, 7.99 Hz, 4H), 7.36 (d, J=8.10 Hz, 2H), 7.23 (d, J=7.72 Hz, 1H), 7.20-7.15 (m, 4H), 5.61 (d, J=11.23 Hz, 1H), 4.63 (d, J=11.21 Hz, 1H), 3.90-3.85 (m, 1H), 3.53-3.48 (m, 1H), 3.37-3.29 (m, 2H), 2.93 (t, J=7.71 Hz, 2H), 2.78-2.61 (m, 2H).

18. (+)-3C-20: C₃₀H₂₄F₆N₂O₃, 574.16, ¹H NMR (600 Hz, Chloroform-d) δ 8.22 (s, 1H), 7.55-7.52 (m, 3H), 7.48 (d, J=8.18 Hz, 2H), 7.38 (d, J=8.18 Hz, 2H), 7.22 (d, J=7.47 Hz, 1H), 7.18-7.12 (m, 4H), 5.67 (d, J=11.20 Hz, 1H), 4.73 (d, J=11.15 Hz, 1H), 3.97-3.92 (m, 1H), 3.56-3.52 (m, 1H), 3.46-3.34 (m, 2H), 2.89 (t, J=7.47 Hz, 2H), 2.75-2.58 (m, 2H).

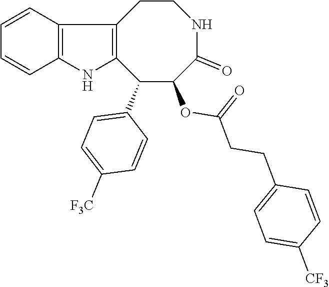 [SMILES: O=C(CCc1ccc(C(F)(F)F)cc1)O[C@@H]1C(=O)NCCc2c([nH]c3ccccc23)[C@H]1c1ccc(C(F)(F)F)cc1]

(±)-3C-20 or (+)-3C-20

Unless otherwise specified, all reagents and materials used in the following examples are commercially available.

### Example 1 In Vitro Screening of Anti-Inflammatory Active Drugs Using a Macrophage Cell Line Inflammation Model

### 1. Experimental Method

RAW264.7 cells were inoculated into a 96-well plate at a density of 1×10⁵ per well. When the cell density was 70%-80%, 18 balasubramide derivatives were prepared into 10 mM working solutions with DMSO, respectively, and then used to pretreat mononuclear macrophages RAW264.7 of mice at a final concentration of 10 μM. After 3 h, lipopolysaccharide (LPS, 100 ng/mL) was added into all wells for action for 2 h except for a control group. A supernatant was collected for later use, and the expression level of an inflammatory factor TNF-α in the cell supernatant was detected according to an operation instruction of a commercial ELISA kit.

### 2. Experimental Results

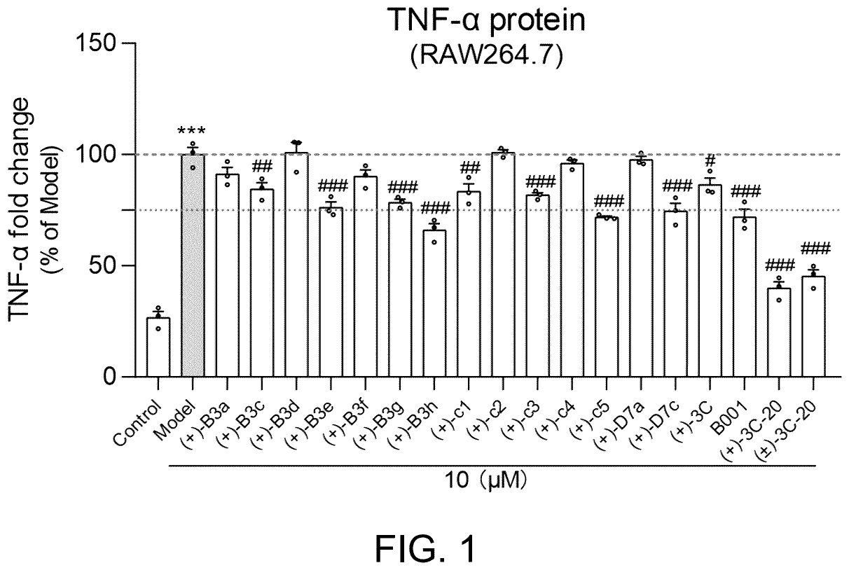

Results are shown in Table 1 and FIG. 1.

TABLE 1
Anti-inflammatory activity of balasubramide derivatives
to a macrophage cell line inflammation model

| | Percentage of TNF- | | Percentage of TNF- |
|---|---|---|---|
| | α in a model group | | α in a model group |
| Group | (%) | Group | (%) |
| Control | 26.74 ± 2.74 | (+)-c2 (10 μM) | 100.96 ± 1.21 |
| Model | 100 ± 3.17*** | (+)-c3 (10 μM) | 81.76 ± 1.11^{###} |
| (+)-B3a (10 μM) | 91.17 ± 3.03 | (+)-c4 (10 μM) | 96.07 ± 1.48 |
| (+)-B3c (10 μM) | 84.48 ± 2.78^{#} | (+)-c5 (10 μM) | 71.77 ± 0.56^{###} |
| (+)-B3d (10 μM) | 100.96 ± 4.46 | (+)-D7a (10 μM) | 97.66 ± 1.5 |
| (+)-B3e (10 μM) | 76.18 ± 2.47^{###} | (+)-D7c (10 μM) | 74.53 ± 3.55^{###} |
| (+)-B3f (10 μM) | 90.17 ± 2.92 | (+)-3C (10 μM) | 86.39 ± 3.06^{#} |
| (+)-B3g (10 μM) | 78.47 ± 1.43^{###} | B001 (10 μM) | 71.93 ± 3.5^{###} |
| (+)-B3h (10 μM) | 66.08 ± 2.9^{###} | (+)-3C-20 (10 μM) | 39.93 ± 2.88^{###} |
| (+)-c1 (10 μM) | 83.41 ± 3.42^{##} | (+)-3C-20 (10 μM) | 45.29 ± 2.89^{###} |

Note:
Compared with the control group,
***P is less than 0.001; and compared with the model group,
^{#}P is less than 0.05,
^{##}P is less than 0.01, and
^{###}P is less than 0.001.

As can be seen from the results in the table and the figure, the lipopolysaccharide can significantly up-regulate the expression of the inflammatory factor TNF-α in the macrophages RAW264.7, and most of the balasubramide derivatives have good anti-inflammatory activity and can well inhibit the expression of the inflammatory factor in the macrophages, in which (+)3C-20 and (±)-3C-20 have better anti-inflammatory activity than other derivatives at a concentration of 10 μM.

### Example 2 Cytotoxicity Test of Balasubramide Derivatives

### 1. Experimental Method

RAW264.7 cells were inoculated into a 96-well plate at a density of 1×10⁵ per well. When the cell density was 70%-80%, the RAW264.7 cells were pretreated with 10 mM working solutions prepared from 18 balasubramide derivatives at a final concentration of 10 μM for 24 h, and then the cell viability was determined by a CCK-8 method.

### 2. Experimental Results

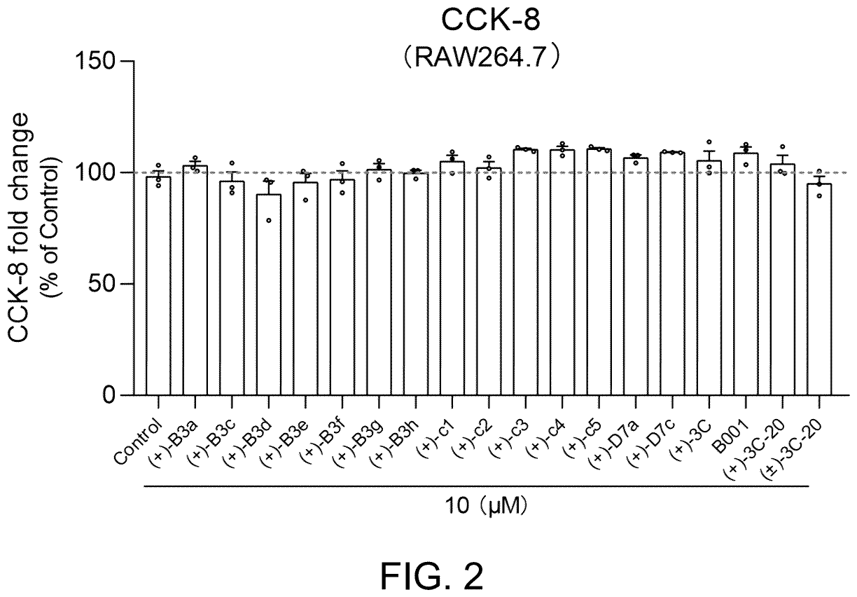

Results are shown in Table 2 and FIG. 2.

TABLE 2
Influence of balasubramide derivatives on the viability of RAW264.7 cells

| | Percentage of CCK- | | Percentage of CCK- |
|---|---|---|---|
| | 8 in a model group | | 8 in a model group |
| Group | (%) | Group | (%) |
| Control | 100 ± 1.16 | (+)-c2 (10 μM) | 102.17 ± 2.74 |
| Model | / | (+)-c3 (10 μM) | 110.41 ± 0.54 |
| (+)-B3a (10 μM) | 103.22 ± 1.79 | (+)-c4 (10 μM) | 110.28 ± 1.56 |
| (+)-B3c (10 μM) | 96.16 ± 4.12 | (+)-c5 (10 μM) | 110.66 ± 0.55 |
| (+)-B3d (10 μM) | 90.33 ± 5.86 | (+)-D7a (10 μM) | 106.69 ± 1.21 |
| (+)-B3e (10 μM) | 95.66 ± 4.02 | (+)-D7c (10 μM) | 109.17 ± 0.16 |
| (+)-B3f (10 μM) | 96.96 ± 3.85 | (+)-3C (10 μM) | 105.39 ± 4.26 |
| (+)-B3g (10 μM) | 101.49 ± 2.57 | B001 (10 μM) | 108.8 ± 2.72 |
| (+)-B3h (10 μM) | 99.75 ± 1.27 | (+)-3C-20 (10 μM) | 103.97 ± 3.88 |
| (+)-c1 (10 μM) | 105.02 ± 2.84 | (+)-3C-20 (10 μM) | 95.04 ± 3.22 |

As can be seen from the results in the table and the figure, all balasubramide derivatives have no obvious toxicity on the RAW264.7 cells at 10 μM.

### Example 3 Determination of the Expression of an Inflammatory Factor in a Macrophage Cell Line

### 1. Experimental Method

RAW264.7 cells were inoculated into a 96-well plate at a density of 1×10⁵ per well. When the cell density was 70%-80%, the RAW264.7 cells were pretreated with (+)3C-20 (0.1-80 M). After 3 h, lipopolysaccharide (LPS, 100 ng/mL) was added into all wells for action for 2 h except for a control group. The expression level of an inflammatory factor TNF-α in a cell supernatant was detected according to an operation instruction of a commercial ELISA kit, and the cell viability at different administration concentrations of (+)3C-20 was determined according to an operation instruction of a CCK-8 kit.

### 2. Experimental Results

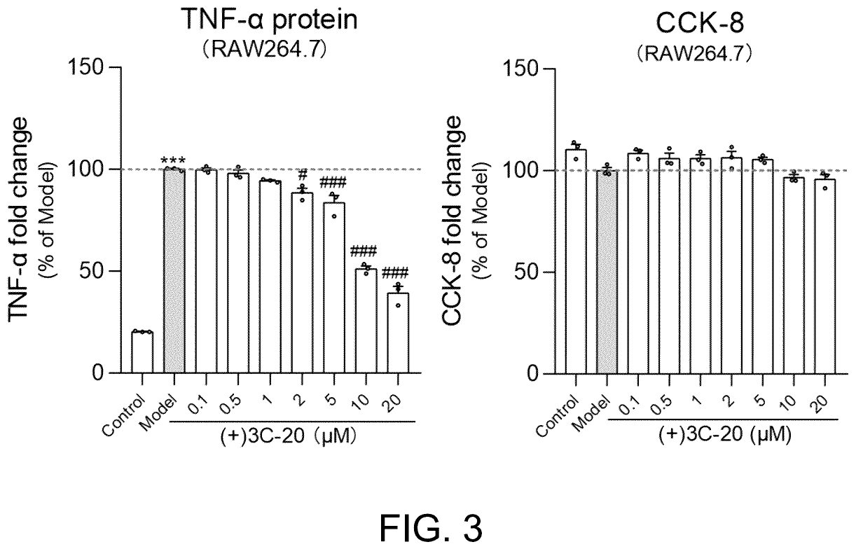

Results are shown in Table 3 and FIG. 3.

TABLE 3
Influence of (+)3C-20 with different concentrations
on TNF-α and cell viability of macrophages

| | Percentage of TNF-α in | Percentage of CCK-8 in |
|---|---|---|
| Group | a model group (%) | a model group (%) |
| Control | 20.36 ± 0.17 | 110.43 ± 2.47 |
| Model | 100 ± 0.43*** | 100 ± 1.53 |
| (+)3C-20 (0.1 μM) | 99.83 ± 0.89 | 108.6 ± 1.49 |
| (+)3C-20 (0.5 μM) | 98.13 ± 1.56 | 106.11 ± 2.44 |
| (+)3C-20 (1 μM) | 94.38 ± 0.45 | 106.01 ± 1.75 |
| (+)3C-20 (2 μM) | 88.59 ± 2.07^{#} | 106.41 ± 3.04 |
| (+)3C-20 (5 μM) | 83.73 ± 3.44^{###} | 105.55 ± 1.06 |
| (+)3C-20 (10 μM) | 51.19 ± 1.33^{###} | 96.64 ± 1.45 |
| (+)3C-20 (20 μM) | 39.35 ± 3.15^{###} | 95.83 ± 2.29 |

Note:
Compared with the control group,
***P is less than 0.001; and compared with the model group,
^{#}P is less than 0.05,
^{##}P is less than 0.01, and
^{###}P is less than 0.001.

As can be seen from the results in the table and the figure, the lipopolysaccharide can significantly up-regulate the expression of the inflammatory factor TNF-α in the macrophages RAW264.7, and the balasubramide derivative (+)3C-20 has good anti-inflammatory activity and can well inhibit the expression of the inflammatory factor TNF-α in the macrophages at an effective dose of 2-20 μM.

### Example 4 Determination of the Expression Level of Inflammation Genes in a Macrophage Cell Line

### 1. Experimental Method

RAW264.7 cells were inoculated into a 24-well plate at a density of 5×10⁵ per well. When the cell density was 70%-80%, the RAW264.7 cells were pretreated with (+)3C-20 (5 μM, 10 μM, 20 μM). After 3 h, lipopolysaccharide (LPS, 100 ng/mL) was added into all wells for action for 2 h except for a control group. A supernatant was discarded, and the cells at the bottom of the plate were fully lysed with Trizol (Nanjing Vazyme, R401-01). RNA was extracted according to an RNA extraction instruction, and subjected to reverse transcription to obtain cDNA, followed by qPCR according to an amplification method at 95° C. for a total of 5 min (at 95° C. for 10 s, and at 60° C. for 30 s) in 40 cycles, and finally statistical calculation was performed by ΔCt.

### 2. Experimental Results

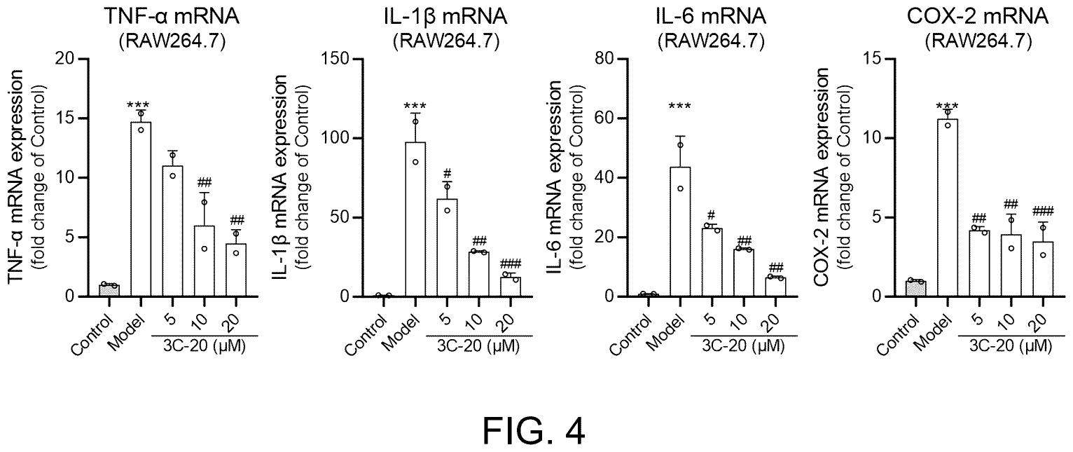

Results are shown in Table 4 and FIG. 4.

TABLE 4
Influence of balasubramide derivatives on the expression
level of inflammation genes in a macrophage cell line

| | Change multiple | Change multiple | Change multiple | Change multiple |
|---|---|---|---|---|
| Group | of TNF-α | of IL-1β | of IL-6 | of COX-2 |
| Control | 1 + 0.07 | 1 ± 0.06 | 1 ± 0 | 1 ± 0.06 |
| Model | 14.72 ± 0.7*** | 97.8 ± 12.78*** | 43.73 ± 7.27*** | 11.23 ± 0.43*** |
| (+)3C-20 | 11.03 ± 0.87 | 62.02 ± 7.52^{#} | 23.17 ± 0.9^{#} | 4.20 ± 0.16^{##} |
| (5 μM) | | | | |
| (+)3C-20 | 6.00 ± 1.96^{##} | 28.51 ± 0.4^{##} | 16.09 ± 0.24^{##} | 3.95 ± 0.9^{##} |
| (10 μM) | | | | |
| (+)3C-20 | 4.49 ± 0.81^{##} | 12.58 ± 1.72^{###} | 6.53 ± 0.31^{##} | 3.49 ± 0.0^{8##} |
| (20 μM) | | | | |

Note:
Compared with the control group,
***P is less than 0.001; and compared with the model group,
^{#}P is less than 0.05,
^{##}P is less than 0.01, and
^{###}P is less than 0.001.

As can be seen from the results in the table and the figure, the lipopolysaccharide can significantly up-regulate the expression of inflammation related genes, including TNF-α, IL-1β, IL-6 and COX-2, in the macrophages RAW264.7, and the balasubramide derivative (+)3C-20 can significantly slow the up-regulation trend after administration and can inhibit the expression of the inflammation genes in a dose-dependent way. That is to say, the (+)3C-20 has good anti-inflammatory activity at an effective dose of 5 μM, 10 μM or 20 μM.

### Example 5 Determination of the Expression of an Inflammatory Factor in a Bone Marrow Derived Macrophage Cell Line

### 1. Experimental Method

(1) Collection of bone marrow derived macrophages (BMDMs): C57BL/6J mice were sacrificed by dislocation of cervical vertebra, completely soaked and sterilized with 75% ethanol and placed faceup on a mouse board to fix the four limbs, the skin of the hind limbs was cut, the limbs were cut at ankle joints and femoral joints, and muscles and knee joints were removed. Precooled serum-free DMEM was sucked with a 1 mL syringe to rinse the bone marrow until a leg bone turned white, a rinsing solution was filtered with a 200-mesh filter membrane and then collected into a 15 mL centrifuge tube, centrifugation was performed at 500×g for 5 min, a supernatant was discarded, and a cell precipitate was resuspended with 1 mL of a red blood cell lysis buffer. After stomatocytosis was performed for 1 min, 5 mL of DMEM was added to stop the stomatocytosis, centrifugation was performed at 500×g for 5 min, a supernatant was discarded, and cells at a lower layer were resuspended overnight with DMEM containing 10% of FBS. Monocytes were collected by centrifugation and then resuspended with a DMEM culture medium containing 20% of FBS, and M-CSF was added at a final concentration of 25 ng/mL. The cells were inoculated into a 96-well plate at a density of 6×10⁵, cultured in a cell incubator with a CO₂ concentration of 5% at 37° C. for 48 h, and then cultured by adding M-CSF with a final concentration of 25 ng/mL into each well for 48 h until the monocytes were subjected to adherent culture and differentiation to obtain macrophages.

(2) Determination of the expression of an inflammatory factor in the bone marrow derived macrophages (BMDMs): The BMDMs were pretreated with (+)3C-20 at a final concentration (0.1 μM, 1 μM, 2.5 μM or 5 μM) for 3 h, and then lipopolysaccharide (LPS, 100 ng/mL) was added into all wells for action for 3 h except for a control group. The expression level of an inflammatory factor TNF-α in a cell supernatant was detected according to an operation instruction of a commercial ELISA kit, and the cell viability at different administration concentrations of (+)3C-20 was determined according to an operation instruction of a CCK-8 kit.

### 2. Experimental Results

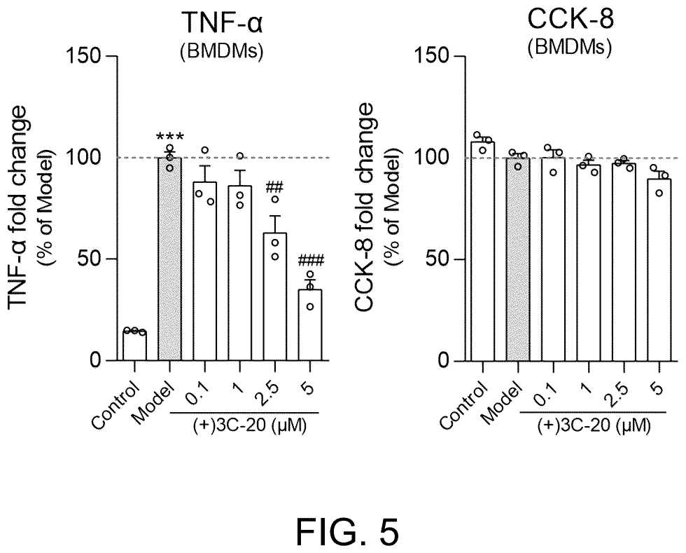

Results are shown in Table 5 and FIG. 5.

TABLE 5
Influence of (+)3C-20 with different concentrations on TNF-α
and cell viability of bone marrow derived macrophages

| | Percentage of TNF-α | Percentage of CCK-8 |
|---|---|---|
| Group | in a model group (%) | in a model group (%) |
| Control | 14.55 ± 0.3 | 108.01 ± 2.25 |
| Model | 100 ± 2.91*** | 100 ± 2.13 |
| (+)3C-20 (0.1 μM) | 88.17 ± 7.91 | 100.35 ± 3.74 |
| (+)3C-20 (1 μM) | 86.35 ± 7.41 | 96.71 ± 2.26 |
| (+)3C-20 (2.5 μM) | 62.94 ± 8.54^{##} | 97.49 ± 1.35 |
| (+)3C-20 (5 μM) | 35.12 ± 4.63^{###} | 89.87 ± 3.68 |

Note:
Compared with the control group,
***P is less than 0.001; and compared with the model group,
^{##}P is less than 0.05, and
^{###}P is less than 0.001.

As can be seen from the results in the table and the figure, the lipopolysaccharide can significantly up-regulate the expression of the inflammatory factor TNF-α in the bone marrow derived macrophages (BMDMs), and the balasubramide derivative (+)3C-20 has good anti-inflammatory activity and can well inhibit the expression of the inflammatory factor TNF-α in the macrophages at an effective dose of 2.5 μM or 5 μM for the BMDMs.

### Example 6 Efficacy Evaluation of (+)3C-20 for Relieving Acute Lung Injury Caused by Sepsis

### 1. Recording of Body Weight and Lung Index

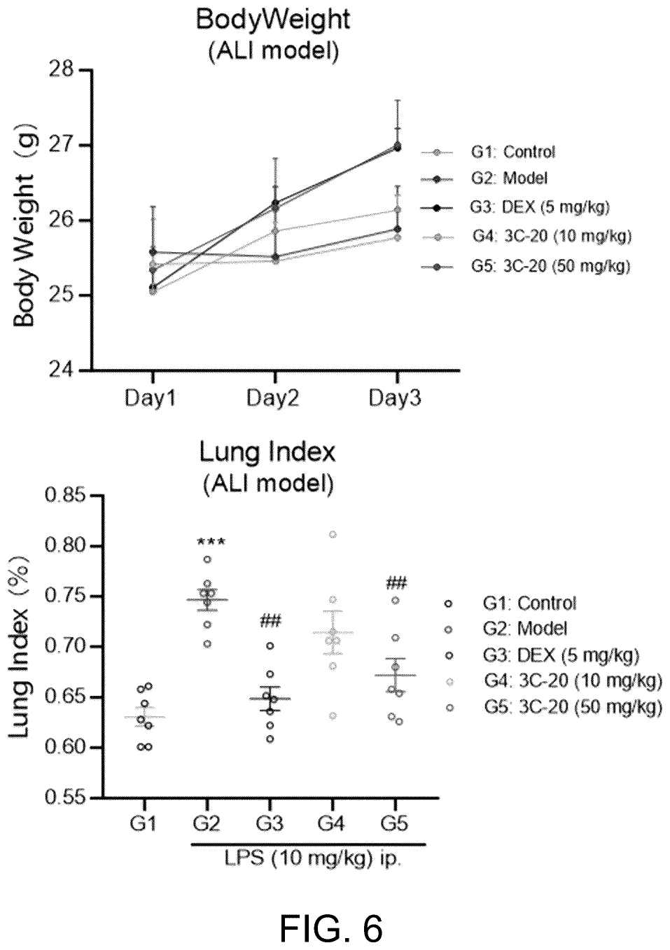

Administration and grouping of animals: Male ICR mice were used as experimental animals, which were randomly divided into a control group, a model group, a positive administration group and administration groups according to the body weight. The control group and the model group were administered with a solvent by intraperitoneal injection (the solvent was prepared from DMSO, Kolliphor HS15 and Saline at a ratio of 1:2:7), the positive administration group was administered with 5 mg/kg dexamethasone (DEX) by intraperitoneal injection, and the administration groups were administered with (+)3C-20 at a concentration of 10 mg/kg and 50 mg/kg, respectively, by intraperitoneal injection. The dexamethasone and the (+)3C-20 were dissolved in the above solvent and were clear and transparent. The administration was continued for 3 days. 2 h after the last administration, all the other groups were administered with LPS (10 mg/kg) by intraperitoneal injection while the control group was administered with an equal volume of normal saline by intraperitoneal injection. Body weight and lung index: The mice were weighed and randomly divided on day 1 and continuously weighed for 3 days before administration, and growth conditions of the mice were measured by statistics. The mice were administered with LPS by intraperitoneal injection 2 h after administration on day 3, then the mice were dissected 6 h later, and the lungs were taken, weighed and recorded. Results are shown in Table 6 and FIG. 6.

TABLE 6
Body weight and lung index of mice

| | Body weight | Body weight | Body weight | Lung index |
|---|---|---|---|---|
| Group | (Day 1) | (Day 2) | (Day 3) | (%) |
| G1: Control | 25.05 ± 0.2 | 25.86 ± 0.21 | 26.14 ± 0.37 | 0.631 ± 0.009 |
| G2: Model | 25.58 ± 1.11 | 25.52 ± 1.13 | 25.89 ± 1.04 | 0.746 ± 0.01*** |
| G3: Dexamethasone (5 mg/kg) | 25.11 ± 0.52 | 26.23 ± 0.38 | 26.97 ± 0.45 | 0.649 ± 0.01^{##} |
| G4: (+)3C-20 (10 mg/kg) | 25.42 ± 1.1 | 25.46 ± 1.2 | 25.77 ± 1.03 | 0.714 ± 0.02 |
| G5: (+)3C-20 (50 mg/kg) | 25.34 ± 0.57 | 26.16 ± 1.22 | 27.01 ± 1.08 | 0.672 ± 0.014^{###} |

Note:
Compared with the control group,
***P is less than 0.001; and compared with the model group,
^{##}P is less than 0.01.

### 2. Determination of Pulmonary Pathology and an Inflammatory Factor in Serum

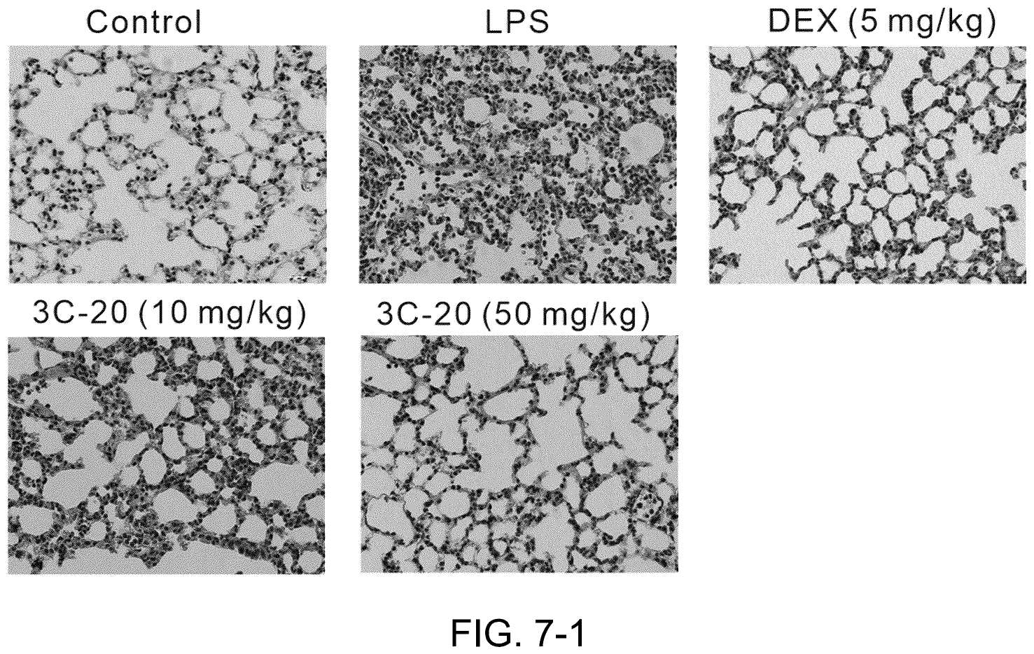

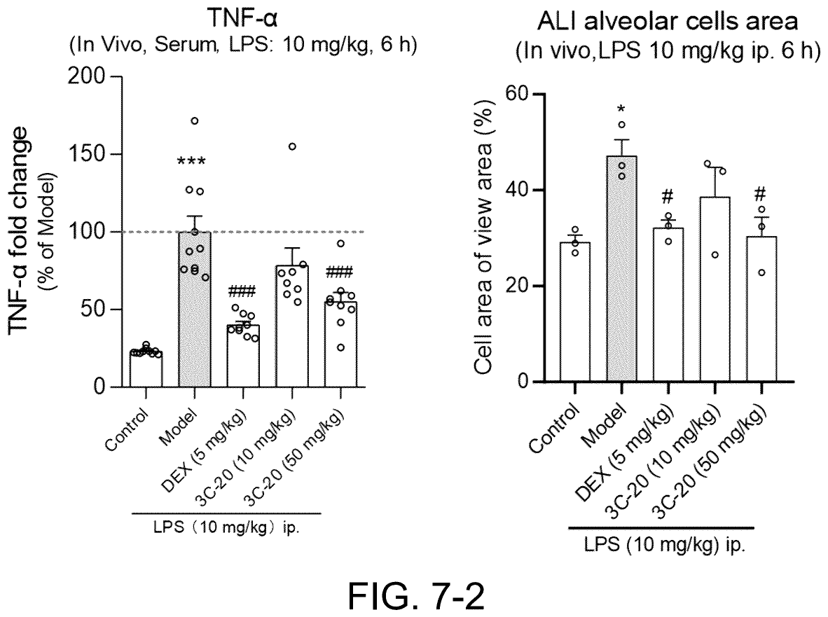

On day 3, 6 h after the intraperitoneal injection of LPS, whole blood was collected from eye sockets and then subjected to centrifugation to prepare serum, and the serum was frozen at −80° C. The lungs were subjected to perfusion with PBS and fixed by paraformaldehyde, and the fixed lungs were stained with hematoxylin-eosin (H&E) after dehydration, paraffin embedding and sectioning. The serum was tested by a commercial TNF-α ELISA kit. Histopathological results and data statistics are shown in FIG. 7-1, FIG. 7-2 and Table 7.

TABLE 7
Data statistics of pulmonary pathology
and an inflammatory factor in serum

| | Area percentage of | Content of TNF-α |
|---|---|---|
| Group | alveolar cells (%) | in serum (% of Model) |
| G1: Control | 29.22 ± 0.87 | 23.11 ± 0.59 |
| G2: Model | 47.24 ± 2.0* | 100 ± 7.19*** |
| G3: Dexamethasone | 32.2 ± 0.95^{#} | 40.09 ± 2.26^{###} |
| (5 mg/kg) | | |
| G4: (+)3C-20 (10 mg/kg) | 38.68 ± 3.73 | 78.38 ± 11.32 |
| G5: (+)3C-20 (50 mg/kg) | 30.43 ± 2.41^{#} | 55.06 ± 5.96^{###} |

Note:
Compared with the control group,
*P is less than 0.05, and
***P is less than 0.001; and compared with the model group,
^{#}P is less than 0.05, and
^{###}P is less than 0.001.

### 3. Determination of the Expression Level of Inflammation Related Genes in Lung Tissues

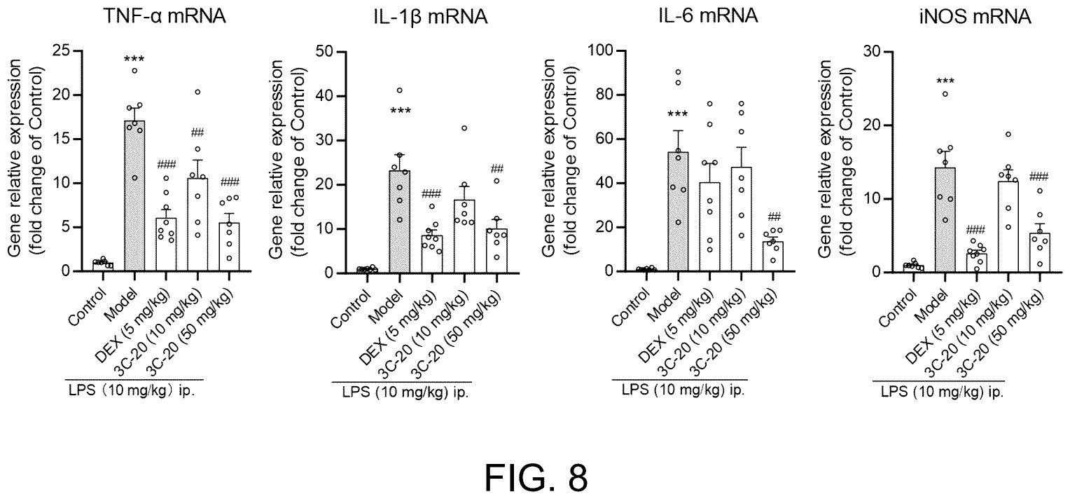

A small amount of frozen lung tissues were taken and fully ground, and the expression of inflammation related genes in the lung tissues was detected by an RT-PCR technology according to an operation instruction of an RNA extraction kit. Experimental results are shown in Table 8 and FIG. 8.

TABLE 8
Expression of inflammation related genes in lung tissues

| | Change multiple | Change multiple | Change multiple | Change multiple |
|---|---|---|---|---|
| Group | of TNF-α | of IL-1β | of IL-6 | of iNOS |
| G1: Control | 1 ± 0.09 | 1 ± 0.07 | 1 ± 0.13 | 1 ± 0.12 |
| G2: Model | 17.14 ± 1.16*** | 23.27 ± 2.96*** | 54.28 ± 8.04*** | 14.31 ± 1.82*** |
| G3: Dexamethasone | 6.09 ± 0.82^{###} | 8.65 ± 1.04^{###} | 40.56 ± 7.55 | 2.61 ± 0.39^{###} |
| (5 mg/kg) | | | | |
| G4: (+)3C-20 | 10.58 ± 1.72^{##} | 9.68 ± 0.67 | 47.5 ± 7.38 | 12.44 ± 1.28 |
| (10 mg/kg) | | | | |
| G5: (+)3C-20 | 5.55 ± 0.86^{###} | 10.13 ± 1.72^{##} | 13.73 ± 1.56^{##} | 5.42 ± 1.02^{###} |
| (50 mg/kg) | | | | |

Note:
Compared with the control group,
***P is less than 0.001; and compared with the model group,
^{#}P is less than 0.05,
^{##}P is less than 0.01, and
^{###}P is less than 0.001.

### 4. Analysis of Results

The body weight of the mice in various administration groups is not changed obviously. 6 h after the intraperitoneal injection of lipopolysaccharide, the lung index of the mice is obviously up-regulated, the content of TNF-α in the serum and the lung tissues is obviously increased, the lung tissues are obviously injured, and the expression level of related inflammation genes in the lung tissues is obviously increased. Compared with the model group, the 3C-20 (50 mg/kg) administration group has the effects of obviously down-regulating the lung index, obviously reducing the expression and release of TNF-α in the serum, reducing the expression of related inflammation genes in the lung tissues and obviously relieving pathological lung injury.

### Example 7 Efficacy Evaluation of (+)3C-20 for Relieving Acute Lung Injury Caused by Lung Exposure to Bacterial Endotoxin

### 1. Recording of Body Weight and Lung Index

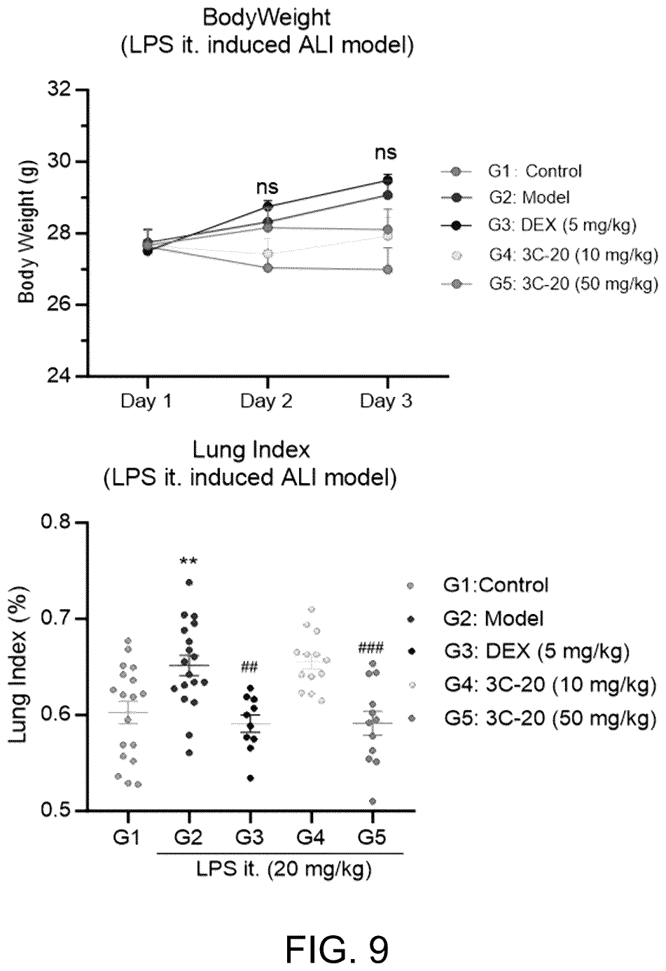

Grouping, administration and modeling of animals: Male ICR mice were used as experimental animals, which were randomly divided into a control group, a model group, a positive administration group and administration groups according to the body weight. The control group and the model group were administered with a solvent by intraperitoneal injection (the solvent was prepared from DMSO, Kolliphor HS15 and Saline at a ratio of 1:2:7), the positive administration group was administered with 5 mg/kg dexamethasone by intraperitoneal injection, and the administration groups were administered with (+)3C-20 at a concentration of 10 mg/kg and 50 mg/kg, respectively, by intraperitoneal injection. The dexamethasone and the (+)3C-20 were dissolved in the above solvent and were clear and transparent. The administration was continued for 3 days. 2 h after the last administration, the mice were anesthetized with 2.5% isoflurane, necks were disinfected, cortices of the necks were cut, and tracheae were separated. All other groups were administered with 50 μL of 10 mg/mL LPS (LPS dissolved in Saline) at a dose of 20 mg/kg by instillation while the control group was administered with 50 μL of Saline by intratracheal instillation (it.), and the cortices were sutured. 6 h after the instillation of LPS, the mice were sacrificed, and serum and lung tissues were taken for later use. Body weight and lung index: The mice were weighed and randomly divided on day 1 and continuously weighed for 3 days before administration. 2 h after the last administration, the mice were administered with 50 μL of 20 mg/mL LPS by intratracheal instillation. 6 h later, the mice were dissected, and the lungs were taken and weighed. Results are shown in Table 9 and FIG. 9.

TABLE 9
Body weight and lung index of mice

| | Body weight | Body weight | Body weight | Lung index |
|---|---|---|---|---|
| Group | (Day 1) | (Day 2) | (Day 3) | (%) |
| G1: Control | 27.67 ± 0.47 | 28.16 ± 0.54 | 28.11 ± 0.57 | 0.603 ± 0.011 |
| G2: Model | 27.75 ± 0.35 | 28.33 ± 0.41 | 29.07 ± 0.38 | 0.651 ± 0.01*** |
| G3: Dexamethasone (5 mg/kg) | 27.51 ± 0.11 | 28.75 ± 0.17 | 29.48 ± 0.17 | 0.591 ± 0.008^{##} |
| G4: (+)3C-20 (10 mg/kg) | 27.67 ± 0.38 | 27.44 ± 0.43 | 27.93 ± 0.53 | 0.656 ± 0.007 |
| G5: (+)3C-20 (50 mg/kg) | 27.63 ± 0.48 | 27.04 ± 0.48 | 27 ± 0.61 | 0.591 ± 0.011^{###} |

Note:
Compared with the control group,
^{ns}P is greater than 0.05, and
***P is less than 0.001; and compared with LPS,
^{##}P is less than 0.01, and
^{###}P is less than 0.001.

### 2. Determination of Pulmonary Pathology and an Inflammatory Factor in Serum

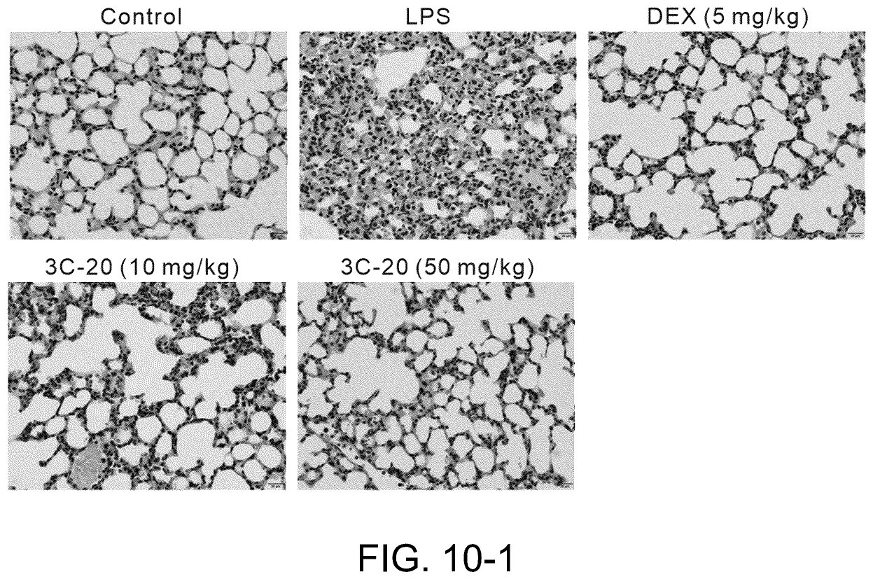

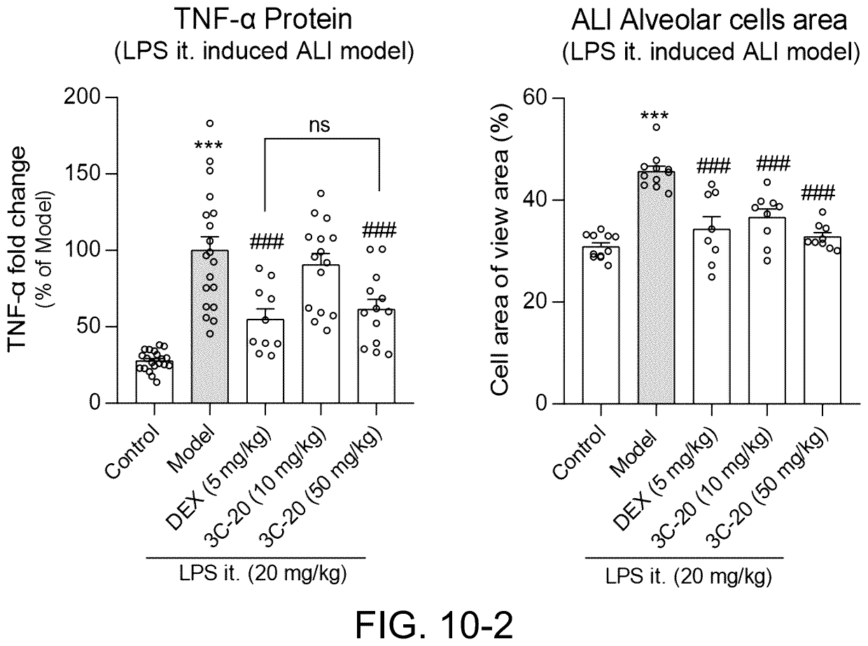

On day 3, 6 h after the intratracheal instillation of LPS, a part of the mice were subjected to perfusion with PBS and treated with paraformaldehyde to fix the right lobe of the lungs, another part of the mice were dissected to obtain lungs, and the lungs were frozen with liquid nitrogen for later use. The fixed right lungs were stained with hematoxylin-eosin (H&E) after dehydration, paraffin embedding and sectioning. 40 mg of the frozen lungs were cut, 300 μL of an RIPA lysis buffer was added thereto and homogenized by starting for 45 s and stopping for 15 s at a frequency of 60 Hz in 4 cycles for full lysis, and then centrifuged at 12,000 rpm at 4° C. A supernatant was taken, the protein concentration was calibrated and adjusted with a BCA protein assay kit, and then the level of an inflammatory factor TNF-α in lung tissue homogenates of various groups was detected by a commercial TNF-α ELISA kit. Results are shown in Table 10, FIG. 10-1 and FIG. 10-2.

TABLE 10
Data statistics of pulmonary pathology
and an inflammatory factor in serum

| | Area percentage of | Content of TNF-α in a |
|---|---|---|
| | alveolar cells | lung tissue homogenate |
| Group | (%) | (% of Model) |
| G1: Control | 30.882 ± 0.731 | 27.337 ± 1.481 |
| G2: Model | 45.641 ± 1.045*** | 100 ± 9.29*** |
| G3: Dexamethasone | 34.322 ± 2.452^{###} | 54.882 ± 6.836^{###} |
| (5 mg/kg) | | |
| G4: (+)3C-20 (10 mg/kg) | 36.611 ± 1.64^{###} | 90.587 ± 7.374 |
| G5: (+)3C-20 (50 mg/kg) | 32.805 ± 0.814^{###} | 61.408 ± 6.525^{###} |

Note:
Compared with the control group,
***P is less than 0.001; and compared with the model group,
^{###}P is less than 0.001.

### 3. Determination of the Expression Level of Inflammation Related Genes in Lung Tissues

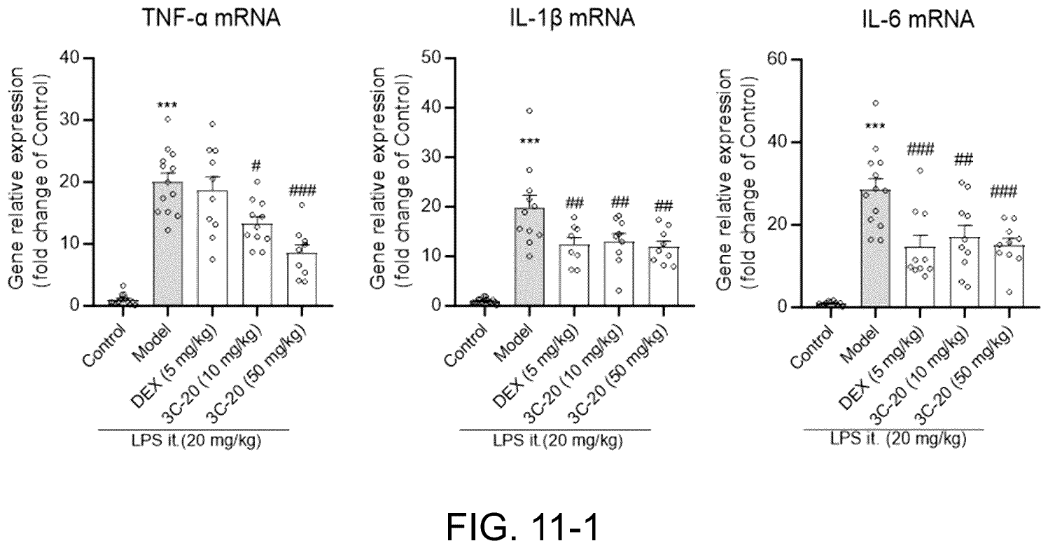

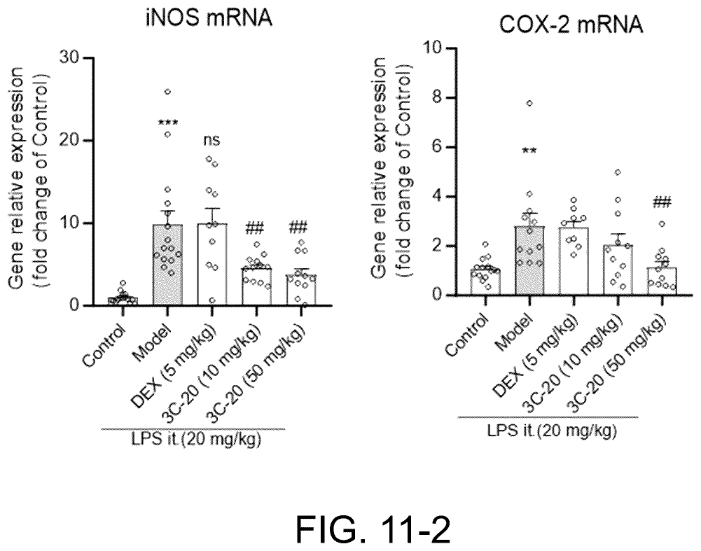

A small amount of frozen lung tissues were taken, a Trizol lysis reagent was added thereto and fully ground, RNA was extracted according to an operation instruction of an RNA extraction kit, and the expression of inflammation related genes in the lung tissues was detected by an RT-PCR technology. Experimental results are shown in Table 11, FIG. 11-1 and FIG. 11-2.

TABLE 11
Expression of inflammation related genes in lung tissues

| | Change multiple | Change multiple | Change multiple | Change multiple | Change multiple |
|---|---|---|---|---|---|
| Group | of TNF-α | of IL-1β | of IL-6 | of iNOS | of COX-2 |
| G1: Control | 1 ± 0.22 | 1 ± 0.13 | 1 ± 0.13 | 1 ± 0.18 | 1.07 ± 0.12 |
| G2: Model | 20.06 ± 1.38*** | 19.87 ± 2.19*** | 28.55 ± 2.55*** | 9.88 ± 1.68*** | 2.8 ± 0.49** |
| G3: | 18.63 ± 2.26 | 12.38 ± 1.28^{##} | 14.75 ± 2.71^{###} | 10.02 ± 1.78 | 2.75 ± 0.24 |
| Dexamethasone | | | | | |
| (5 mg/kg) | | | | | |
| G4: (+)3C-20 | 13.32 ± 0.94^{#} | 13.04 ± 1.24^{##} | 17.12 ± 2.28^{##} | 4.56 ± 0.4^{##} | 2.05 ± 0.38 |
| (10 mg/kg) | | | | | |
| G5: (+)3C-20 | 8.56 ± 1.21^{###} | 11.94 ± 1.05^{##} | 15.11 ± 1.58^{###} | 3.75 ± 0.72^{##} | 1.14 ± 0.24^{##} |
| (50 mg/kg) | | | | | |

Note:
Compared with the control group,
***P is less than 0.001; and compared with the model group,
^{#}P is less than 0.05,
^{##}P is less than 0.01, and
^{###}P is less than 0.001.

### 4. Analysis of Results

The body weight of the mice in various administration groups is not changed obviously. 6 h after the intratracheal instillation of lipopolysaccharide, the lung index of the mice is obviously up-regulated, the content of TNF-α in the lung tissues is obviously increased, the lung tissues are obviously injured, and the expression level of related inflammation genes in the lung tissues is obviously increased. Compared with the model group, the (+)3C-20 (50 mg/kg) administration group has the effects of obviously down-regulating the lung index, obviously reducing the expression and release of TNF-α in the lung tissues, reducing the expression of related inflammation genes in the lung tissues and obviously relieving pathological lung injury.

### Example 8 Relieving Effect of (+)3C-20 on Acute Lung Injury Caused by Intratracheal Instillation of Lipopolysaccharide

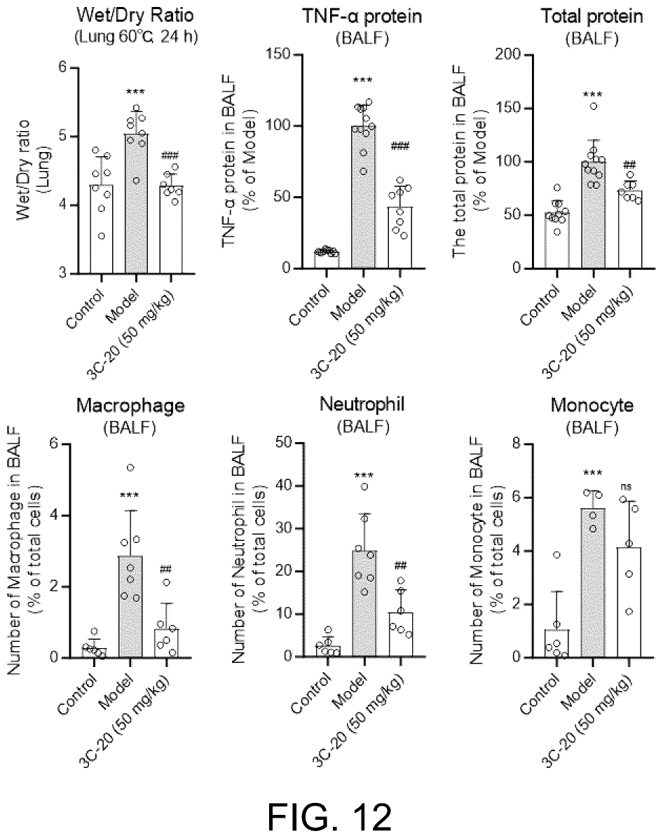

Grouping and administration of animals: 50 male ICR mice were randomly divided into 3 groups according to the body weight, including a control group, a model group and a 50 mg/kg (+)3C-20 group. Drug preparation, administration and a modeling method were the same as those in Example 7. After continuous preadministration for 3 days, intratracheal instillation of LPS was performed for modeling 2 h after the last administration. 6 h after the modeling, a part of the mice were directly sacrificed, the whole lungs were taken out and weighed to obtain wet weight, then the whole lungs were dried at 60° C. for 24 h and weighed to obtain dry weight, and the wet-dry weight ratio of each group was calculated by statistics. 6 h after the modeling, the remaining mice were subjected to bronchoalveolar lavage, and then bronchoalveolar lavage fluid (BALF) was collected. A lavage process was as follows: After being sacrificed, the mice were placed faceup to fix the four limbs, the skin of the neck was cut, the trachea was separated and cut at an appropriate position to obtain a small incision, a 16G needle with a flat tip was inserted into the trachea at a depth of 1 cm, the trachea and the needle stem were tied tightly to obtain an indwelling needle, 400 μL of precooled PBS was sucked with a 1 mL syringe and slowly pushed into the trachea, suction was performed for two times, the operations above were repeated, and recovered BALF was combined with a recovery efficiency of 70%-80%. The collected BALF was subjected to centrifugation at 500×g at 4° C. for 5 min, a supernatant was collected, the content level of total protein in the supernatant was determined by a BCA kit, and the expression level of an inflammatory factor TNF-α in the BALF supernatant was detected according to an operation instruction of a commercial ELISA kit. Results are shown in Table 12 and FIG. 12.

A bottom precipitate obtained after the centrifugation of the BALF was resuspended with 100 μL of an ACK buffer (Thermo A10492 stomatocytosis agent), evenly blown and subjected to standing for 1 min. Stomatocytosis was stopped with 1 mL of an FACS buffer (PBS containing 1% of FBS), centrifugation was performed at 500×g at 4° C. for 5 min, a supernatant was discarded, 100 μL of an FACS buffer was added, 1 μL of a CD16/CD32 Fc Block antibody (1:100) was added, and incubation was performed in the dark at 4° C. for 30 min. 1 mL of an FACS buffer was added, centrifugation was performed at 500×g at 4° C. for 5 min, a supernatant was discarded, and 100 μL of an FACS buffer was added for resuspension. Various fluorescent dyes (including mCD45-PerCP-Cy5.5, mCD11b-FITC, mF4/80-APC, mLy6C-PE, mLy6G-BV421 and FVS-780) were added at a ratio of 1:300, a single dye tube of each of the fluorescent dyes was prepared according to the above ratio, and incubation was performed in the dark at 4° C. for 30 min. 1 mL of an FACS buffer was added for resuspension, centrifugation was performed at 500×g at 4° C. for 5 min, and a supernatant was discarded. Washing was repeated for one time, centrifugation was performed to remove a supernatant, 500 μL of 1% paraformaldehyde was added for fixing in the dark for 30 min, centrifugation was performed at 500×g at 4° C. for 5 min, and a supernatant was discarded. 200 μL of an FACS buffer was added for resuspension, and a resulting suspension was stored in the dark at 4° C. Before loading on a machine, the stained cell suspension was diluted with an FACS buffer, transferred to a flow tube and detected according to an operation procedure of a CytoFlex flow cytometer. A flow gate strategy was as follows: Gain was first regulated according to each single dye tube, and compensation was properly regulated, where CD45⁺, CD11b⁺ and F4/80⁺ were macrophages, CD45⁺, CD11b⁺ and Ly6G⁺ were neutrophils, and CD45⁺, CD11b⁺ and Ly6C⁺ were monocytes. Results are shown in Table 12 and FIG. 12.

TABLE 12
Relieving effect of (+)3C-20 on acute lung injury caused
by intratracheal instillation of lipopolysaccharide

| | | | Level of | | | |
|---|---|---|---|---|---|---|
| | | Level of | total | | | |
| | Wet/Dry | TNF-α in | protein in | Macrophage | Neutrophil | Monocyte |
| Group | ratio | BALF | BALF | (%) | (%) | (%) |
| Control | 4.3 ± | 11.92 ± | 52.71 ± | 0.29 ± | 2.64 ± | 1.06 ± |
| | 0.15 | 0.34 | 3.55 | 0.1 | 0.85 | 0.59 |
| Model | 5.04 ± | 100 ± | 100 ± | 2.87 ± | 24.86 ± | 5.61 ± |
| | 0.12*** | 0.04*** | 6.12*** | 0.48*** | 3.28*** | 0.25*** |
| (+)3C-20 | 4.28 ± | 43.31 ± | 73.44 ± | 0.82 ± | 10.47 ± | 4.13 ± |
| (50 mg/kg) | 0.06^{###} | 5.19^{###} | 3.09^{##} | 0.27^{##} | 2.13^{##} | 0.66 |

Note:
Compared with the control group,
***P is less than 0.001; and compared with the model group,
^{#}P is less than 0.05,
^{##}P is less than 0.01, and
^{###}P is less than 0.001.

The experimental results show that 6 h after the intratracheal instillation of lipopolysaccharide, the lung wet/dry weight ratio of the mice is obviously up-regulated, the levels of TNF-α and total protein in the BALF is obviously increased, and the contents of infiltrated macrophages, neutrophils and monocytes in the BALF are also obviously up-regulated. Compared with the model group, the (+)3C-20 (50 mg/kg) administration group can obviously down-regulate the lung wet/dry weight ratio of the mice, down-regulate the levels of total protein and TNF-α in the BALF and reduce the infiltration of macrophages, neutrophils and monocytes.

### Example 9 Influence of (+)3C-20 on the Survival Rate of Mice with Acute Lung Injury Caused by Intratracheal Instillation of Lipopolysaccharide

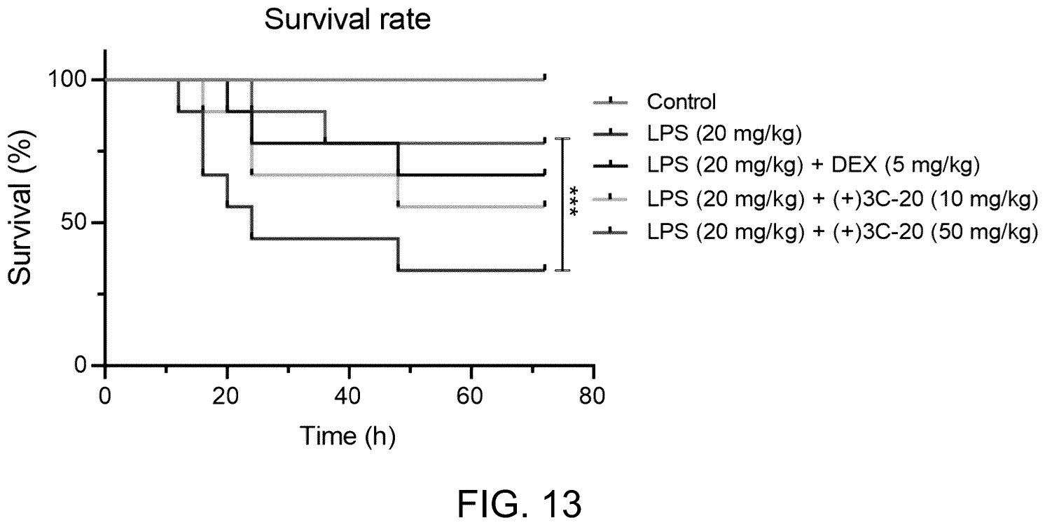

Grouping and administration of animals: 45 male ICR mice were randomly divided into 5 groups according to the body weight, including a control group, a model group, a positive dexamethasone (5 mg/kg) group, a (+)3C-20 (10 mg/kg) group and a (+)3C-20 (50 mg/kg) group, with 9 mice in each group. Drug preparation, an administration method and a modeling method were the same as those in Example 7. With the time of intratracheal instillation of LPS as 0 h, the survival state of the mice was checked at the time points of 4 h, 12 h, 16 h, 20 h, 24 h, 36 h, 48 h, 60 h and 72 h, and a survival curve was drawn based on statistics. Results are shown in FIG. 13 (where compared with LPS (20 mg/kg), ***P is less than 0.001).

As can be seen from the figure, 50 mg/kg (+)3C-20 can obviously improve the survival rate of the model mice with acute lung injury induced by LPS (20 mg/kg it).

The above examples are preferred embodiments of the present invention, but the embodiments of the present invention are not limited by the above examples. Any other changes, modifications, substitutions, combinations and simplifications that are made without deviating from the spirit, essence and principles of the present invention shall be equivalent replacement ways, which shall be included in the scope of protection of the present invention.

What is claimed is:

1. A balasubramide derivative, wherein the balasubramide derivative has a structure shown in Formula (I): 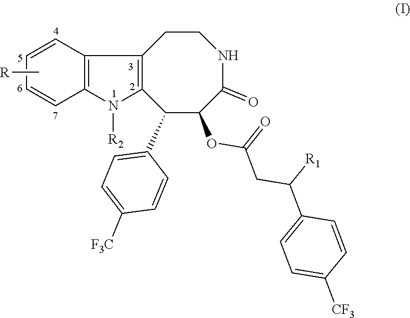
wherein R is hydrogen, halogen, C₁₋₄ alkyl, C₁₋₇ alkoxyl, benzyloxy or nitro-substituted benzoyloxy; R₁ is hydrogen or 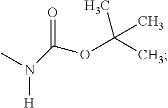
R₂ is hydrogen, C₁₋₄ alkyl or C₂₋₅ alkenyl; and R, R₁ and R₂ are not hydrogen simultaneously.

2. The balasubramide derivative according to claim 1, wherein R is hydrogen, halogen, C₁₋₃ alkyl, C₁₋₅ alkoxyl, benzyloxy or nitro-substituted benzoyloxy; R₁ is hydrogen or 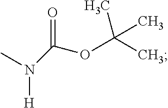
R₂ is hydrogen, C₁₋₃ alkyl or C₂₋₃ alkenyl; and R, R₁ and R₂ are not hydrogen simultaneously.

3. The balasubramide derivative according to claim 2, wherein R is hydrogen, halogen, methyl, ethoxyl, benzyloxyl or nitro-substituted benzoyloxy; R₁ is hydrogen or 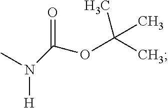
R₂ is hydrogen, methyl, ethyl, propyl or allyl; and R, R₁ and R₂ are not hydrogen simultaneously.

4. Use of a balasubramide derivative in preparation of an anti-inflammatory medicine, wherein the balasubramide derivative has a structure shown in Formula (I): 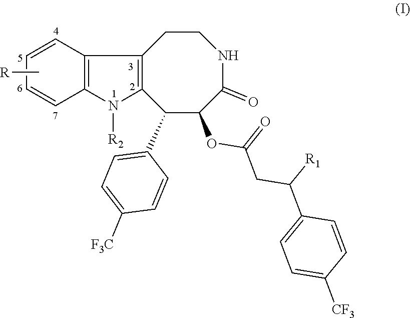
wherein R is hydrogen, halogen, C₁₋₄ alkyl, C₁₋₇ alkoxyl, benzyloxy or nitro-substituted benzoyloxy; R₁ is hydrogen or 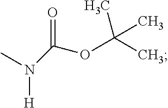
and R₂ is hydrogen, C₁₋₄ alkyl or C₂₋₅ alkenyl.

5. Use of a balasubramide derivative in preparation of a medicine for treating acute lung injury, wherein the balasubramide derivative has a structure shown in Formula (I): 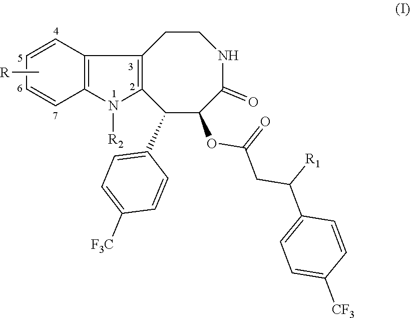
wherein R is hydrogen, halogen, C₁₋₄ alkyl, C₁₋₇ alkoxyl, benzyloxy or nitro-substituted benzoyloxy; R₁ is hydrogen or 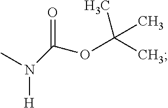
and R₂ is hydrogen, C₁₋₄ alkyl or C₂₋₅ alkenyl.

6. The use according to claim 5, wherein the acute lung injury is caused by sepsis.

7. The use according to claim 5, wherein the acute lung injury is induced by bacterial endotoxin.

8. The use according to claim 5, wherein the acute lung injury comprises septicopyemia caused by peripheral inflammation, inflammatory lung injury, bronchial asthma, tracheitis, bronchitis, chronic obstructive pulmonary disease, pulmonary heart disease and pulmonary fibrosis.

9. The use according to claim 4, wherein the medicine comprises an effective amount of the balasubramide derivative or a pharmaceutically acceptable salt, solvate, enantiomer, diastereoisomer or tautomer thereof.

10. The use according to claim 4, wherein the medicine has a dosage form of a pulmonary or nasal inhalation nebulizer, a quantitative aerosol or a dry powder inhaler.

11. The use according to claim 6, wherein the acute lung injury comprises septicopyemia caused by peripheral inflammation, inflammatory lung injury, bronchial asthma, tracheitis, bronchitis, chronic obstructive pulmonary disease, pulmonary heart disease and pulmonary fibrosis.

12. The use according to claim 7, wherein the acute lung injury comprises septicopyemia caused by peripheral inflammation, inflammatory lung injury, bronchial asthma, tracheitis, bronchitis, chronic obstructive pulmonary disease, pulmonary heart disease and pulmonary fibrosis.

13. The use according to claim 5, wherein the medicine comprises an effective amount of the balasubramide derivative or a pharmaceutically acceptable salt, solvate, enantiomer, diastereoisomer or tautomer thereof.

14. The use according to claim 5, wherein the medicine has a dosage form of a pulmonary or nasal inhalation nebulizer, a quantitative aerosol or a dry powder inhaler.
